(19) United States
(12) Patent Application Publication
(10) Pub. No.: US 2025/0090654 A1
(43) Pub. Date: Mar. 20, 2025

(54) CORONAVIRUS VACCINE COMPOSITIONS AND USES THEREOF

(71) Applicant: BOOST BIOPHARMA, INC., Amherst, MA (US)

(72) Inventors: Fritz Schomburg, Madison, WI (US); David Rancour, Madison, WI (US)

(73) Assignee: BOOST BIOPHARMA, INC., Amherst, MA (US)

(21) Appl. No.: 18/725,577

(22) PCT Filed: Dec. 30, 2022
(86) PCT No.: PCT/US2022/082665

§ 371 Date: Jun. 28, 2024

Related U.S. Application Data
(60) Provisional application No. 63/295,782, filed on Dec. 31, 2021.

(51) Int. Cl. A61K 39/215; C07K 14/005

(52) U.S. Cl. CPC A61K 39/215; C07K 14/005; C07K 2319/30; C12N 2770/20022; C12N 2770/20034

(57) ABSTRACT

Provided is a recombinant polypeptide containing at least one immunogenic fragment of Severe Acute Respiratory Syndrome Coronavirus 2 (SARS-CoV-2) spike glycoprotein. Also provided are a method for preventing, inhibiting, reducing, eliminating, protecting, or delaying the onset of an infection or an infectious clinical condition caused by a coronavirus in a subject which includes administering to the subject the recombinant polypeptide, and a method for inducing an immune response against a coronavirus in a subject, which includes administering to the subject the recombinant polypeptide.

### CROSS-REFERENCE TO PRIOR APPLICATION

This application claims benefit to U.S. Provisional Patent Application No. 63/295,782, filed Dec. 31, 2021, which is hereby incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 2,422,598 Byte XML file named “SEQL_766400,” created on Dec. 30, 2022.

### BACKGROUND OF THE INVENTION

The continued rapid evolution of new SARS-CoV-2 variants containing mutations that alter the amino acid sequence of the Spike protein resulting in altered function, and altered resistance to native immune defenses and to immune defenses elicited by currently marketed vaccines has led to a need for alternative platforms that allow for incorporation of new variant mutations in a robust, adaptable, biologically active, and cost-effective vaccine.

### BRIEF SUMMARY OF THE INVENTION

A recombinant polypeptide comprising at least one immunogenic fragment of Severe Acute Respiratory Syndrome Coronavirus 2 (SARS-CoV-2) spike glycoprotein, wherein each immunogenic fragment comprises an N-terminus region, wherein the N-terminus region comprises an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355. In some embodiments, each immunogenic fragment of the recombinant polypeptide further comprises a C-terminus region, wherein the C-terminus region comprises an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423.

In some embodiments, each immunogenic fragment further comprises an intervening region between the N-terminus region and the C-terminus region, wherein each intervening region comprises an amino acid sequence selected from the group consisting of SEQ ID NOs: 2045-2050 or that differs at at least 4 residues from an amino acid sequence selected from the group consisting of SEQ ID NOs: 603-606 and 2037-2040.

Additional embodiments include, but are not limited to, pharmaceutical compositions comprising the recombinant polypeptide and a pharmaceutically acceptable carrier, and methods of using the same.

### BRIEF DESCRIPTION OF THE DRAWINGS

FIG. 1 is a schematic depicting certain fragments of the SARS-CoV-2 receptor binding domain, namely CTD_shortg (SEQ ID NO: 35), CTD-short_d (SEQ ID NO: 29), CTD_short_h (SEQ ID NO: 37), CTD_short_i (SEQ ID NO:39), CTD_vs_a (SEQ ID NO: 1) and an exemplary intervening region sequence according to an embodiment of the invention (SEQ ID NO: 603).

FIG. 2A is an SDS-PAGE gel photograph depicting CHO cell expression of the listed constructs expressing certain immunogenic fragments after 5 days. Protein was affinity purified using Protein A agarose from equal volumes of transfected culture. Eluted protein was loaded based on volume, separated by reducing SDS-PAGE, and detected by Coomassie R-250 staining. Load volumes were twice as large for samples (left to right) CTD_vs_a-Fc to RBD_e-Fc compared to the samples on the left of the gel (CTD_long_b-Fc to CTD_short_h-Fc). Expression of CTD_short_g demonstrates protease sensitivity, whereas CTD_short_h, CTD_short_d, and CTD_short_i do not exhibit this sensitivity.

FIG. 2B is an SDS-PAGE gel photograph depicting CHO cell expression of CTD_short_a-Fc (LS2330) after 5 days. Protein was affinity purified using Protein A agarose and analyzed by reducing SDS-PAGE and detected by Coomassie R-250 staining. M-Molecular weight markers. This construct has very poor expression in terms of yield and protease sensitivity.

FIG. 2C is an SDS-PAGE gel photograph depicting CHO cell expression of CTD_short_i-Fc (LS2371) after 5 days, demonstrating robust expression with minimal protease sensitivity. Protein was affinity purified using Protein A agarose and analyzed by reducing SDS-PAGE and detected by Coomassie R-250 staining.

FIG. 3As, 3B, 3C, and 3D are SDS-PAGE gel photographs depicting CHO cell expression of sequential N-terminal deletion mutants of CTD-short-g (CTD-g). Construct naming nomenclature (N-#) indicate the terminus from which the deletion was made and the number of amino acids deleted (anywhere between −1 and −47). “M” is marker. Gel samples are presented left to right with increasing number of deleted amino acids. Full-length protein is seen as the primary band at about 55-60 kDa. A dimer of the protein (an in vitro result of high concentration of purified protein) is found at about 120 kDa.

FIGS. 4A and 4B are graphs depicting the expression of sequential N-terminal deletion mutants of CTD-short-g (CTD-g) as measured from samples depicted in FIGS. 3A-3D, wherein FIG. 4A depicts absolute expression and FIG. 4B depicts expression relative to CTD-g.

FIGS. 5A, 5B, and 5C are SDS-PAGE gel photographs depicting CHO cell expression of sequential C-terminal deletion mutants of CTD-short-g (CTD-g). Construct naming nomenclature (C-#) indicate the terminus from which the deletion was made and the number of amino acids deleted (anywhere between −1 and −35). “M” denotes a marker. Gel samples are presented left to right with increasing number of deleted amino acids. Full-length protein is seen as the primary band at about 55-60 kDa. A dimer of the protein (an in vitro result of high concentration of purified protein) is found at about 120 kDa.

FIGS. 6A and 6B are graphs depicting the expression of sequential C-terminal deletion mutants of CTD-short-g (CTD-g) as measured from samples depicted in FIGS. 5A-5C, wherein FIG. 6A depicts absolute expression and FIG. 6B depicts expression relative to CTD-g.

FIG. 7 is a set of SDS-PAGE gel photographs depicting expression of certain immunogenic fragment constructs in CHO-S cells or in ExpiCHO cells. “M1” and “M2” denote markers. Full-length protein is seen as the primary band at about 55-60 kDa. A dimer of the protein (an in vitro result of high concentration of purified protein) is found at about 120 kDa. Degraded protein can be seen at about 38 kDa with respect to CTD-short_g grown in CHO-S cells.

FIGS. 8A, 8B, 8C, 8D, and 8E are SDS-PAGE gel photographs depicting CHO cell expression of certain immunogenic fragments. Fragments are combinations of previously determined N- and C-terminal deletions as indicated in the names, N-# and C-#, where the # is the number of amino acids deleted from the indicated end from CTD-short-g sequence. “M” denotes the marker. Full-length protein is seen as the primary band at about 55-60 kDa. A dimer of the protein (an in vitro result of high concentration of purified protein) is found at about 120 kDa.

### DETAILED DESCRIPTION OF THE INVENTION

Recombinant polypeptides of the invention can include any suitable immunogenic fragment or fragments of the Severe Acute Respiratory Syndrome Coronavirus 2 (SARS-CoV-2) spike glycoprotein as described herein.

### Description of Immunogenic Fragments

Immunogenic fragments according to embodiments of the invention are described herein. In certain embodiments, immunogenic fragments include certain regions which are also described herein. For instance, in some embodiments, an immunogenic fragment of SARS-CoV-2 spike glycoprotein comprises an N-terminus region. In some embodiments, an immunogenic fragment of SARS-CoV-2 spike glycoprotein comprises an N-terminus region and a C-terminus region. In some embodiments, an immunogenic fragment of SARS-CoV-2 spike glycoprotein comprises an N-terminus region, an intervening region, and a C-terminus region. In some embodiments, an immunogenic fragment of SARS-CoV-2 spike glycoprotein comprises an N-terminus region, an intervening region that further comprises an N-terminus-adjacent region and/or a C-terminus-adjacent region, and a C-terminus region. Unless otherwise stated, such immunogenic fragment regions can be combined together in any suitable fashion to form immunogenic fragments in accordance with the description herein. In some embodiments, the immunogenic fragments are recombinant and/or isolated.

In some embodiments, an immunogenic fragment of SARS-CoV-2 spike glycoprotein comprises an N-terminus region and a C-terminus region, wherein the N-terminus region comprises, consists of, or consists essentially of, an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355. In some embodiments, the N-terminus region comprises, consists of, or consists essentially of, an amino acid sequence selected from the group consisting of SEQ ID NOs: 245-248, 293-296, 314-317 and 342-345. In some embodiments, the N-terminus region comprises, consists of, or consists essentially of, an amino acid sequence selected from the group consisting of SEQ ID NOs: 250-251, 298-299, 319-320, and 347-348. In some embodiments, the N-terminus region comprises, consists of, or consists essentially of, an amino acid sequence selected from the group consisting of SEQ ID NOs: 255-261, 303-309, 324-330, and 352-358. In some embodiments, the N-terminus region comprises, consists of, or consists essentially of, an amino acid sequence selected from the group consisting of SEQ ID NOs: 256-258, 304-306, 325-327, and 353-355.

In some embodiments, the N-terminus region comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355. In some embodiments, the amino acid sequence is selected from the group consisting of SEQ ID NOs: 245-248, 293-296, 314-317 and 342-345. In some embodiments, the amino acid sequence is selected from the group consisting of SEQ ID NOs: 250-251, 298-299, 319-320, and 347-348. In some embodiments, the amino acid sequence is selected from the group consisting of SEQ ID NOs: 255-261, 303-309, 324-330, and 352-358. In some embodiments, the amino acid sequence is selected from the group consisting of SEQ ID NOs: 256-258, 304-306, 325-327, and 353-355. In some embodiments, the N-terminus region amino acid sequence differs at 10 residues. In some embodiments, the N-terminus region amino acid sequence differs at 9 residues. In some embodiments, the N-terminus region amino acid sequence differs at 8 residues. In some embodiments, the N-terminus region amino acid sequence differs at 7 residues. In some embodiments, the N-terminus region amino acid sequence differs at 5 residues. In some embodiments, the N-terminus region amino acid sequence differs at 4 residues. In some embodiments, the N-terminus region amino acid sequence differs at 3 residues. In some embodiments, the N-terminus region amino acid sequence differs at 2 residues. In some embodiments, the N-terminus region amino acid sequence differs at 1 residue.

In some embodiments, the N-terminus region comprises, consists of, or consists essentially of, an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, wherein the total length of the amino acid sequence of the immunogenic fragment, including the N-terminus region, is 203-227 amino acid residues, preferably 203-210, 211-215, 216-220, or 221-227 residues, more preferably 205-210 or 207 residues.

With regard to regions of immunogenic fragments, immunogenic fragments themselves, and polypeptides containing such fragments, when an amino acid sequence differs or diverges from another amino acid sequence, the differing amino acid residues can be any suitable mutation, including conservative and non-conservative amino acid mutations. Suitable mutations can also include amino acid deletions and additions. For instance, a conservative substitution can replace one aliphatic amino acid (i.e., Glycine, Alanine, Valine, Leucine, Methionine or Isoleucine) for another, one polar, uncharged R group amino acid (i.e., Serine, Cysteine, Threonine, Proline, Asparagine, or Methionine) for another, one positively charged R group amino acid (i.e., Histidine, Lysine, or Arginine) for another, one negatively charged R group amino acid (i.e., Aspartate or Glutamate) for another, and one non-polar, aromatic R group amino acid (i.e., Phenylalanine, Tyrosine, or Tryptophan) for another.

In some embodiments, the N-terminus region comprises, consists of, or consists essentially of, an amino acid sequence that differs at no residues or at least 1, at least 2, at least 3, at least 4, at least 5, at least 6, at least 7, at least 8, at least 9, at least 10, at least 11, at least 12, at least 13, at least 14, or at least 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355. In some embodiments, the amino acid sequence is selected from the group consisting of SEQ ID NOs: 245-248, 293-296, 314-317 and 342-345. In some embodiments, the amino acid sequence is selected from the group consisting of SEQ ID NOs: 250-251, 298-299, 319-320, and 347-348. In some embodiments, the amino acid sequence is selected from the group consisting of SEQ ID NOs: 255-261, 303-309, 324-330, and 352-358. In some embodiments, the amino acid sequence is selected from the group consisting of SEQ ID NOs: 256-258, 304-306, 325-327, and 353-355. In some embodiments, the N-terminus region amino acid sequence differs at 10 residues. In some embodiments, the N-terminus region amino acid sequence differs at least 9 residues. In some embodiments, the N-terminus region amino acid sequence differs at least 8 residues. In some embodiments, the N-terminus region amino acid sequence differs at least 7 residues. In some embodiments, the N-terminus region amino acid sequence differs at least 5 residues. In some embodiments, the N-terminus region amino acid sequence differs at least 4 residues. In some embodiments, the N-terminus region amino acid sequence differs at least 3 residues. In some embodiments, the N-terminus region amino acid sequence differs at least 2 residues. In some embodiments, the N-terminus region amino acid sequence differs at least 1 residue. In some embodiments, the N-terminus region comprises, consists of, or consists essentially of, an amino acid sequence that differs at no more than 1, no more than 2, no more than 3, no more than 4, no more than 5, no more than 6, no more than 7, no more than 8, no more than 9, no more than 10, no more than 11, no more than 12, no more than 13, no more than 14, no more than 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355. In some embodiments, the amino acid sequence is selected from the group consisting of SEQ ID NOs: 245-248, 293-296, 314-317 and 342-345. In some embodiments, the amino acid sequence is selected from the group consisting of SEQ ID NOs: 250-251, 298-299, 319-320, and 347-348. In some embodiments, the amino acid sequence is selected from the group consisting of SEQ ID NOs: 255-261, 303-309, 324-330, and 352-358. In some embodiments, the amino acid sequence is selected from the group consisting of SEQ ID NOs: 256-258, 304-306, 325-327, and 353-355. In some embodiments, the N-terminus region amino acid sequence differs at no more than 10 residues. In some embodiments, the N-terminus region amino acid sequence differs at no more than 9 residues. In some embodiments, the N-terminus region amino acid sequence differs at no more than 8 residues. In some embodiments, the N-terminus region amino acid sequence differs at no more than 7 residues. In some embodiments, the N-terminus region amino acid sequence differs at no more than 5 residues. In some embodiments, the N-terminus region amino acid sequence differs at no more than 4 residues. In some embodiments, the N-terminus region amino acid sequence differs at no more than 3 residues. In some embodiments, the N-terminus region amino acid sequence differs at no more than 2 residues. In some embodiments, the N-terminus region amino acid sequence differs at no more than 1 residue.

In some embodiments, the N-terminus region comprises, consists of, or consists essentially of, an amino acid sequence which differs at a numerical range of residues from an amino acid sequence selected from the group consisting of SEQ ID NO: 245-289 and 293-362, and amino acid sequences DYS, YS, and S preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355. In some embodiments, the amino acid sequence is selected from the group consisting of SEQ ID NOs: 245-248, 293-296, 314-317 and 342-345. In some embodiments, the amino acid sequence is selected from the group consisting of SEQ ID NOs: 250-251, 298-299, 319-320, and 347-348. In some embodiments, the amino acid sequence is selected from the group consisting of SEQ ID NOs: 255-261, 303-309, 324-330, and 352-358. In some embodiments, the amino acid sequence is selected from the group consisting of SEQ ID NOs: 256-258, 304-306, 325-327, and 353-355. In such embodiments, the minimum number of differing residues is any value from the “at least” list recited in the previous paragraph, e.g., 4 residues, and the maximum number of differing residues is any value from the “no more than” list recited in the previous paragraph, e.g., 8 residues, as long as the bottom range value is less than the top range value. For example, a range of differing residues can be 2-5 residues. In some embodiments, the range of differing residues is 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4- 5, 5-10, −5-9, 5-8, 5-7, 5-6, 6-10, 6-9, 6-8, 6-7, 7-10, 7-9, 7-8, 8-10, 8-9, 9-10, 0-10, 0-1, 0-2, 0-3, 0-4, 0-5, 0-6, 0-7, 0-8, 0-9, or 0-10.

The term “no more than” when used to reference a number of differing residues, includes no residues as well. For example, when a first amino acid sequence differs at no more than two residues from a second amino acid sequence, there can 0, 1 or 2 differing residues between the two sequences.

In some embodiments, an immunogenic fragment of SARS-CoV-2 spike glycoprotein comprises an N-terminus region and a C-terminus region, wherein the C-terminus region comprises, consists of, or consists essentially of an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423. In some embodiments, the amino acid sequence is selected from SEQ ID NOs: 382-397. In some embodiments, the amino acid sequence is selected from SEQ ID NOs: 382-387 and 421-423. In some embodiments, the amino acid sequence is selected from SEQ ID NOs: 388-393. In some embodiments, the amino acid sequence is selected from SEQ ID NOs: 394-397. In some embodiments, the amino acid sequence is selected from SEQ ID NOs: 385-390. In some embodiments, the amino acid sequence is selected from SEQ ID NOs: 391-395.

In some embodiments, an immunogenic fragment of SARS-CoV-2 spike glycoprotein comprises an N-terminus region and a C-terminus region, wherein the C-terminus region comprises, consists of, or consists essentially of an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423, wherein the total length of the amino acid sequence of the immunogenic fragment, including the N- and C-terminus regions, is 203-227 amino acid residues, preferably 203-210, 211-215, 216-220, or 221-227 residues, more preferably 205-210 or 207 residues.

In some embodiments, the C-terminus region comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423. In some embodiments, the amino acid sequence is selected from SEQ ID NOs: 382-397. In some embodiments, the amino acid sequence is selected from SEQ ID NOs: 382-387 and 421-423. In some embodiments, the amino acid sequence is selected from SEQ ID NOs: 388-393. In some embodiments, the amino acid sequence is selected from SEQ ID NOs: 394-397. In some embodiments, the amino acid sequence is selected from SEQ ID NOs: 385-390. In some embodiments, the amino acid sequence is selected from SEQ ID NOs: 391-395. In some embodiments, the C-terminus region amino acid sequence differs at 10 residues. In some embodiments, the C-terminus region amino acid sequence differs at 9 residues. In some embodiments, the C-terminus region amino acid sequence differs at 8 residues. In some embodiments, the C-terminus region amino acid sequence differs at 7 residues. In some embodiments, the C-terminus region amino acid sequence differs at 5 residues. In some embodiments, the C-terminus region amino acid sequence differs at 4 residues. In some embodiments, the C-terminus region amino acid sequence differs at 3 residues. In some embodiments, the C-terminus region amino acid sequence differs at 2 residues. In some embodiments, the C-terminus region amino acid sequence differs at 1 residue.

In some embodiments, the C-terminus region comprises, consists of, or consists essentially of, an amino acid sequence that differs at no residues or at least 1, at least 2, at least 3, at least 4, at least 5, at least 6, at least 7, at least 8, at least 9, at least 10, at least 11, at least 12, at least 13, at least 14, or at least 15 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423. In some embodiments, the amino acid sequence is selected from SEQ ID NOs: 382-397. In some embodiments, the amino acid sequence is selected from SEQ ID NOs: 382-387 and 421-423. In some embodiments, the amino acid sequence is selected from SEQ ID NOs: 388-393. In some embodiments, the amino acid sequence is selected from SEQ ID NOs: 394-397. In some embodiments, the amino acid sequence is selected from SEQ ID NOs: 385-390. In some embodiments, the amino acid sequence is selected from SEQ ID NOs: 391-395. In some embodiments, the C-terminus region amino acid sequence differs at 10 residues. In some embodiments, the C-terminus region amino acid sequence differs at least 9 residues. In some embodiments, the C-terminus region amino acid sequence differs at least 8 residues. In some embodiments, the C-terminus region amino acid sequence differs at least 7 residues. In some embodiments, the C-terminus region amino acid sequence differs at least 5 residues. In some embodiments, the C-terminus region amino acid sequence differs at least 4 residues. In some embodiments, the C-terminus region amino acid sequence differs at least 3 residues. In some embodiments, the C-terminus region amino acid sequence differs at least 2 residues. In some embodiments, the C-terminus region amino acid sequence differs at least 1 residue. In some embodiments, the C-terminus region comprises, consists of, or consists essentially of, an amino acid sequence that differs at no more than 1, no more than 2, no more than 3, no more than 4, no more than 5, no more than 6, no more than 7, no more than 8, no more than 9, no more than 10, no more than 11, no more than 12, no more than 13, no more than 14, or no more than 15 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423. In some embodiments, the amino acid sequence is selected from SEQ ID NOs: 382-397. In some embodiments, the amino acid sequence is selected from SEQ ID NOs: 382-387 and 421-423. In some embodiments, the amino acid sequence is selected from SEQ ID NOs: 388-393. In some embodiments, the amino acid sequence is selected from SEQ ID NOs: 394-397. In some embodiments, the amino acid sequence is selected from SEQ ID NOs: 385-390. In some embodiments, the amino acid sequence is selected from SEQ ID NOs: 391-395. In some embodiments, the C-terminus region amino acid sequence differs at no more than 10 residues. In some embodiments, the C-terminus region amino acid sequence differs at no more than 9 residues. In some embodiments, the C-terminus region amino acid sequence differs at no more than 8 residues. In some embodiments, the C-terminus region amino acid sequence differs at no more than 7 residues. In some embodiments, the C-terminus region amino acid sequence differs at no more than 5 residues. In some embodiments, the C-terminus region amino acid sequence differs at no more than 4 residues. In some embodiments, the C-terminus region amino acid sequence differs at no more than 3 residues. In some embodiments, the C-terminus region amino acid sequence differs at no more than 2 residues. In some embodiments, the C-terminus region amino acid sequence differs at no more than 1 residue.

In some embodiments, the C-terminus region comprises, consists of, or consists essentially of, an amino acid sequence which differs at a numerical range of residues from an amino acid sequence selected from the group consisting of SEQ ID NO: 363-398 and 402-423, and amino acid sequences STN, ST, and S, preferably selected from SEQ ID NOs: 382-397, 382-387, 388-393, 394-397, 385-390, 391-395, and 421-423. In some embodiments, the amino acid sequence is selected from SEQ ID NOs: 382-397. In some embodiments, the amino acid sequence is selected from SEQ ID NOs: 382-387 and 421-423. In some embodiments, the amino acid sequence is selected from SEQ ID NOs: 388-393. In some embodiments, the amino acid sequence is selected from SEQ ID NOs: 394-397. In some embodiments, the amino acid sequence is selected from SEQ ID NOs: 385-390. In some embodiments, the amino acid sequence is selected from SEQ ID NOs: 391-395. In such embodiments, the minimum number of differing residues is any value from the “at least” list recited in the previous paragraph, e.g., 4 residues, and the maximum number of differing residues is any value from the “no more than” list recited in the previous paragraph, e.g., 6 residues, as long as the bottom range value is less than the top range value. For example, a range of differing residues can be 4-7. In some embodiments, the range of differing residues is 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, −5-9, 5-8, 5-7, 5-6, 6-10, 6-9, 6-8, 6-7, 7-10, 7-9, 7-8, 8-10, 8-9, 9-10, 0-10, 0-1, 0-2, 0-3, 0-4, 0-5, 0-6, 0-7, 0-8, 0-9, or 0-10.

In some embodiments, the immunogenic fragment of SARS-CoV-2 spike glycoprotein comprises an N-terminus region and a C-terminus region, wherein the N-terminus region comprises an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and the C-terminus region comprises an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423. In some embodiments, the N-terminus amino acid sequence is selected from the group consisting of SEQ ID NOs: 245-248, 293-296, 314-317 and 342-345. In some embodiments, the N-terminus amino acid sequence is selected from the group consisting of SEQ ID NOs: 250-251, 298-299, 319-320, and 347-348. In some embodiments, the N-terminus amino acid sequence is selected from the group consisting of SEQ ID NOs: 255-261, 303-309, 324-330, and 352-358. In some embodiments, the N-terminus amino acid sequence is selected from the group consisting of SEQ ID NOs: 256-258, 304-306, 325-327, and 353-355. In some embodiments, the C-terminus amino acid sequence is selected from SEQ ID NOs: 382-397. In some embodiments, the C-terminus amino acid sequence is selected from SEQ ID NOs: 382-387 and 421-423. In some embodiments, the C-terminus amino acid sequence is selected from SEQ ID NOs: 388-393. In some embodiments, the C-terminus amino acid sequence is selected from SEQ ID NOs: 394-397. In some embodiments, the C-terminus amino acid sequence is selected from SEQ ID NOs: 385-390. In some embodiments, the C-terminus amino acid sequence is selected from SEQ ID NOs: 391-395. Any combination of N-terminus region and C-terminus region sequences can be used in an immunogenic fragment, for example, an N-terminus region comprising an amino acid sequence of SEQ ID NO: 258 and a C-terminus region comprising an amino acid sequence of SEQ ID NO: 395. In some embodiments, the total length of the amino acid sequence of the immunogenic fragment, including the N- and C-terminus regions, is 203-227 amino acid residues, preferably 203-210, 211-215, 216-220, or 221-227 residues, more preferably 205-210 or 207 residues.

In certain embodiments, an immunogenic fragment according to the invention further comprises an intervening region located between the N-terminus region and the C-terminus region. The intervening region can comprise, consist of, or consist essentially of, any suitable amino acid sequence.

In some embodiments, the intervening region comprises, consists of, or consists essentially of, an amino acid sequence with at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 603-606, 2037-2040, and 2045-2050. In some embodiments, the intervening region comprises, consists of, or consists essentially of, an amino acid sequence with no more than 70%, no more than 71%, no more than 72%, no more than 73%, no more than 74%, no more than 75%, no more than 76%, no more than 77%, no more than 78%, no more than 79%, no more than 80%, no more than 81%, no more than 82%, no more than 83%, no more than 84%, no more than 85%, no more than 86%, no more than 87%, no more than 88%, no more than 89%, no more than 90%, no more than 91%, no more than 92%, no more than 93%, no more than 94%, no more than 95%, no more than 96%, no more than 97%, no more than 98%, or no more than 99% identity to an amino acid sequence selected from the group consisting of SEQ ID NO: 603-606, 2037-2040, and 2045-2050. In one embodiment, the amino acid sequence has no more than 90% identity. In one embodiment, the amino acid sequence has no more than 91% identity. In one embodiment, the amino acid sequence has no more than 92% identity. In one embodiment, the amino acid sequence has no more than 93% identity. In one embodiment, the amino acid sequence has no more than 94% identity. In one embodiment, the amino acid sequence has no more than 95% identity. In one embodiment, the amino acid sequence has no more than 96% identity. In one embodiment, the amino acid sequence has no more than 97% identity. In one embodiment, the amino acid sequence has no more than 98% identity. In one embodiment, the amino acid sequence has no more than 99% identity.

In some embodiments, the intervening region comprises, consists of, or consists essentially of, an amino acid sequence with a range of percent identity to an amino acid sequence selected from the group consisting of SEQ ID NO: 603-606, 2037-2040, and 2045-2050, wherein the percent identity value for the bottom of the range is any value from the “at least” list of values recited in the previous paragraph, e.g., 60%, and the percent identity value for the top of the range is any value from “no more than” list recited in the previous paragraph, e.g., 88%, as long as the bottom range value is less than the top range value. For example, a range of percent identity can be between at least 84% and 98% identity. In one embodiment, the range is 90-99, 90-98, 90-97, 90-96, 90-95, 90-94, 90-93, 90-92, 90-91, 91-99, 91-98, 91-97, 91-96, 91-95, 91-94, 91-93, 91-92, 92-99, 92-98, 92-97, 92-96, 92-95, 92-94, 92-93, 93-99, 93-98, 93-97, 93-96, 93-95, 93-94, 94-99, 94-98, 94-97, 94-96, 94-95, 95-99, 95-98, 95-97, 95-96, 96-99, 96-98, 96-97, 97-99, 97-98, or 98-99.

In some embodiments, the intervening region comprises, consists of, or consists essentially of, an amino acid sequence that differs at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues from an amino acid sequence selected from the group consisting of SEQ ID NOs: 603-606, 2037-2040, and 2045-2050. In some embodiments, the intervening region amino acid sequence differs at 20 residues. In some embodiments, the intervening region amino acid sequence differs at 19 residues. In some embodiments, the intervening region amino acid sequence differs at 18 residues. In some embodiments, the intervening region amino acid sequence differs at 17 residues. In some embodiments, the intervening region amino acid sequence differs at 16 residues. In some embodiments, the intervening region amino acid sequence differs at 15 residues. In some embodiments, the intervening region amino acid sequence differs at 14 residues. In some embodiments, the intervening region amino acid sequence differs at 13 residues. In some embodiments, the intervening region amino acid sequence differs at 12 residues. In some embodiments, the intervening region amino acid sequence differs at 11 residues. In some embodiments, the intervening region amino acid sequence differs at 10 residues. In some embodiments, the intervening region amino acid sequence differs at 9 residues. In some embodiments, the intervening region amino acid sequence differs at 8 residues. In some embodiments, the intervening region amino acid sequence differs at 7 residues. In some embodiments, the intervening region amino acid sequence differs at 5 residues. In some embodiments, the intervening region amino acid sequence differs at 4 residues. In some embodiments, the intervening region amino acid sequence differs at 3 residues. In some embodiments, the intervening region amino acid sequence differs at 2 residues. In some embodiments, the intervening region amino acid sequence differs at 1 residue.

In some embodiments, the intervening region comprises, consists of, or consists essentially of, an amino acid sequence that differs at no residues or at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 residues from an amino acid sequence selected from the group consisting of SEQ ID NOs: 603-606, 2037-2040, and 2045-2050. In some embodiments, the intervening region amino acid sequence differs at at least 20 residues. In some embodiments, the intervening region amino acid sequence differs at at least 19 residues. In some embodiments, the intervening region amino acid sequence differs at at least 18 residues. In some embodiments, the intervening region amino acid sequence differs at at least 17 residues. In some embodiments, the intervening region amino acid sequence differs at at least 16 residues. In some embodiments, the intervening region amino acid sequence differs at at least 15 residues. In some embodiments, the intervening region amino acid sequence differs at at least 14 residues. In some embodiments, the intervening region amino acid sequence differs at at least 13 residues. In some embodiments, the intervening region amino acid sequence differs at at least 12 residues. In some embodiments, the intervening region amino acid sequence differs at at least 11 residues. In some embodiments, the intervening region amino acid sequence differs at at least 10 residues. In some embodiments, the intervening region amino acid sequence differs at at least 9 residues. In some embodiments, the intervening region amino acid sequence differs at at least 8 residues. In some embodiments, the intervening region amino acid sequence differs at at least 7 residues. In some embodiments, the intervening region amino acid sequence differs at at least 5 residues. In some embodiments, the intervening region amino acid sequence differs at at least 4 residues. In some embodiments, the intervening region amino acid sequence differs at at least 3 residues. In some embodiments, the intervening region amino acid sequence differs at at least 2 residues. In some embodiments, the intervening region amino acid sequence differs at at least 1 residue.

In some embodiments, the intervening region comprises, consists of, or consists essentially of, an amino acid sequence that differs at no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 21, no more than 22, no more than 23, no more than 24, no more than 25, no more than 26, no more than 27, no more than 28, no more than 29, no more than 30, no more than 31, no more than 32, no more than 33, no more than 34, no more than 35, no more than 36, no more than 37, no more than 38, no more than 39, no more than 40, no more than 41, no more than 42, no more than 43, no more than 44, no more than 45, no more than 46, no more than 47, no more than 48, no more than 49, or no more than 50 residues from an amino acid sequence selected from the group consisting of SEQ ID NO: 603-606, 2037-2040, and 2045-2050. In some embodiments, the intervening region amino acid sequence differs at no more than 20 residues. In some embodiments, the intervening region amino acid sequence differs at no more than 19 residues. In some embodiments, the intervening region amino acid sequence differs at no more than 18 residues. In some embodiments, the intervening region amino acid sequence differs at no more than 17 residues. In some embodiments, the intervening region amino acid sequence differs at no more than 16 residues. In some embodiments, the intervening region amino acid sequence differs at no more than 15 residues. In some embodiments, the intervening region amino acid sequence differs at no more than 14 residues. In some embodiments, the intervening region amino acid sequence differs at no more than 13 residues. In some embodiments, the intervening region amino acid sequence differs at no more than 12 residues. In some embodiments, the intervening region amino acid sequence differs at no more than 11 residues. In some embodiments, the intervening region amino acid sequence differs at no more than 10 residues. In some embodiments, the intervening region amino acid sequence differs at no more than 9 residues. In some embodiments, the intervening region amino acid sequence differs at no more than 8 residues. In some embodiments, the intervening region amino acid sequence differs at no more than 7 residues. In some embodiments, the intervening region amino acid sequence differs at no more than 5 residues. In some embodiments, the intervening region amino acid sequence differs at no more than 4 residues. In some embodiments, the intervening region amino acid sequence differs at no more than 3 residues. In some embodiments, the intervening region amino acid sequence differs at no more than 2 residues. In some embodiments, the intervening region amino acid sequence differs at no more than 1 residue.

In some embodiments, the intervening region comprises, consists of, or consists essentially of, an amino acid sequence with a range of residues at which the sequence differs from an amino acid sequence selected from the group consisting of SEQ ID NO: 603-606, 2037-2040, and 2045-2050, wherein the minimum number of differing residues is any value from the “at least” list recited above two paragraphs previous, e.g., 4 residues, and the maximum number of differing residues is any value from the “no more than” list recited in the previous paragraph, e.g., 33 residues, as long as the bottom range value is less than the top range value. For example, a range of differing residues can be 2-8 residues. In some embodiments, the range of differing residues is 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, −5-9, 5-8, 5-7, 5-6, 6-10, 6-9, 6-8, 6-7, 7-10, 7-9, 7-8, 8-10, 8-9, 9-10, 0-10, 0-1, 0-2, 0-3, 0-4, 0-5, 0-6, 0-7, 0-8, 0-9, or 0-10.

In some immunogenic fragment embodiments of the invention, the intervening region is directly adjacent to one or both of the N-terminus and C-terminus regions, i.e., there are no amino acid residues between the amino acid sequences of the N-terminus region and the intervening region, there are no amino acid residues between the amino acid sequences of the C-terminus region and the intervening region, or there are no amino acid residues between either the amino acid sequences of the N-terminus region and the intervening region, or between the amino acid sequences of the C-terminus and the intervening region. In other embodiments, one or more amino acid residues are present between the amino acid sequences of the N-terminus region and the intervening region, between the amino acid sequences of the C-terminus and the intervening region, or both. When one or more amino acid residues are present, there can be, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 35, 36, 37, 38, 39, 40, 41, 42, 43, 44, 45, 46, 47, 48, 49, 50, or more amino acid residues present between an nearest residue of the N- or C-terminus region and the intervening region.

In some embodiments, the intervening region further comprises an N-terminus-adjacent region, a C-terminus-adjacent region, or both. Non-limiting examples of N-terminus-adjacent and C-terminus-adjacent regions are respectively shown in Tables 1 and 2 below. In some embodiments, as can be seen in Table 1, wherein the N-terminus region amino acid sequence is selected from the amino acid sequences of SEQ ID NOs: 611-668, in most cases there are one or more corresponding N-terminus-adjacent region amino acid sequences, as shown in the table below. In some embodiments, as can be seen in Table 2, wherein the C-terminus region amino acid sequence is selected from the amino acid sequences of SEQ ID NOs: 669-712, in most cases there are one or more corresponding C-terminus-adjacent region amino acid sequences as shown in the table below.

TABLE 1

| N-terminus Region | | Corresponding N-terminus- |
|---|---|---|
| | SEQ ID | adjacent region(s) |
| Name | NO: | SEQ ID NO(s) or AA sequence: |
| CTD-gN-5mer-end-d00 | 611 | 815 or 969 or 1001 or 1047 |
| CTD-gN-5mer-end-d01 | 612 | 816 or 970 or 1002 or 1048 |
| CTD-gN-5mer-end-d02 | 613 | 817 or 971 or 1003 or 1049 |
| CTD-gN-5mer-end-d03 | 614 | 818 or 972 or 1004 or 1050 |
| CTD-gN-5mer-end-d04 | 615 | 819 or 973 or 1005 or 1051 |
| CTD-gN-5mer-end-d05 | 616 | 820 or 974 or 1006 or 1052 |
| CTD-gN-5mer-end-d06 | 617 | 821 or 975 or 1007 or 1053 |
| CTD-gN-5mer-end-d07 | 618 | 822 or 976 or 1008 or 1054 |
| CTD-gN-5mer-end-d08 | 619 | 823 or 977 or 1009 or 1055 |
| CTD-gN-5mer-end-d09 | 620 | 824 or 978 or 1010 or 1056 |
| CTD-gN-5mer-end-d10 | 621 | 825 or 979 or 1011 or 1057 |
| CTD-gN-5mer-end-d11 | 622 | 826 or 980 or 1012 or 1058 |
| CTD-gN-5mer-end-d12 | 623 | 827 or 981 or 1013 or 1059 |
| CTD-gN-5mer-end-d13 | 624 | 828 or 982 or 1014 or 1060 |
| CTD-gN-5mer-end-d14 | 625 | 829 or 983 or 1015 or 1061 |
| CTD-gN-5mer-end-d15 | 626 | 830 or 984 or 1016 or 1062 |
| CTD-gN-5mer-end-d16 | 627 | 831 or 1017 |
| CTD-gN-5mer-end-d17 | 628 | 832 or 1018 |
| CTD-gN-5mer-end-d18 | 629 | 833 or 1019 |
| CTD-gN-5mer-end-d19 | 630 | 834 or 1020 |
| CTD-gN-5mer-end-d20 | 631 | 835 or 1021 |
| CTD-gN-5mer-end-d21 | 632 | 836 or 1022 |
| CTD-gN-5mer-end-d22 | 633 | 837 or 1023 |
| CTD-gN-5mer-end-d23 | 634 | 838 |
| CTD-gN-5mer-end-d24 | 635 | 839 |
| CTD-gN-5mer-end-d25 | 636 | 840 |
| CTD-gN-5mer-end-d26 | 637 | 841 |
| CTD-gN-5mer-end-d27 | 638 | 842 |
| CTD-gN-5mer-end-d28 | 639 | 843 |
| CTD-gN-5mer-end-d29 | 640 | 844 |
| CTD-gN-5mer-end-d30 | 641 | 845 |
| CTD-gN-5mer-end-d31 | 642 | 846 |
| CTD-gN-5mer-end-d32 | 643 | 847 |
| CTD-gN-5mer-end-d33 | 644 | 848 |
| CTD-gN-5mer-end-d34 | 645 | 849 |
| CTD-gN-5mer-end-d35 | 646 | 850 |
| CTD-gN-5mer-end-d36 | 647 | 851 |
| CTD-gN-5mer-end-d37 | 648 | 852 |
| CTD-gN-5mer-end-d38 | 649 | 853 |
| CTD-gN-5mer-end-d39 | 650 | 854 |
| CTD-gN-5mer-end-d40 | 651 | DYS |
| CTD-gN-5mer-end-d41 | 652 | YS |
| CTD-gN-5mer-end-d42 | 653 | S |
| CTD-gN-5mer-end-d43 | 654 | N/A |
| CTD-gN-5mer-end-d44 | 655 | N/A |
| CTD-gN-5mer-end-d45 | 656 | N/A |
| CTD-gN-5mer-end-d46 | 657 | N/A |
| CTD-gN-5mer-end-d47 | 658 | N/A |
| CTD-gN-5mer-end- | 659 | 831 or 1017 |
| d16_Om | | |
| CTD-gN-5mer-end- | 660 | 832 or 1018 |
| d17_Om | | |
| CTD-gN-5mer-end- | 661 | 833 or 1019 |
| d18_Om | | |
| CTD-gN-5mer-end- | 662 | 834 or 1020 |
| d19_Om | | |
| CTD-gN-5mer-end- | 663 | 835 or 1021 |
| d20_Om | | |
| CTD-gN-5mer-end- | 664 | 838 |
| d23_Mu | | |
| CTD-gN-5mer-end- | 665 | 839 |
| d24_Mu | | |
| CTD-gN-5mer-end- | 666 | 840 |
| d25_Mu | | |
| CTD-gN-5mer-end- | 667 | 841 |
| d26_Mu | | |
| CTD-gN-5mer-end- | 668 | 842 |
| d27_Mu | | |

TABLE 2

| C-terminus Region | | Corresponding C-terminus- |
|---|---|---|
| | SEQ ID | adjacent region(s) |
| Name | NO: | SEQ ID NO(s) or AA sequence: |
| CTD-gC-5mer-end-d00 | 669 | 901 or 1079 |
| CTD-gC-5mer-end-d01 | 670 | 902 or 1080 |
| CTD-gC-5mer-end-d02 | 671 | 903 or 1081 |
| CTD-gC-5mer-end-d03 | 672 | 904 or 1082 |
| CTD-gC-5mer-end-d04 | 673 | 905 or 1083 |
| CTD-gC-5mer-end-d05 | 674 | 906 or 1084 |
| CTD-gC-5mer-end-d06 | 675 | 907 or 1085 |
| CTD-gC-5mer-end-d07 | 676 | 908 or 1086 |
| CTD-gC-5mer-end-d08 | 677 | 909 or 1087 |
| CTD-gC-5mer-end-d09 | 678 | 910 or 1088 |
| CTD-gC-5mer-end-d10 | 679 | 911 or 1089 |
| CTD-gC-5mer-end-d11 | 680 | 912 or 1090 |
| CTD-gC-5mer-end-d12 | 681 | 913 or 1091 |
| CTD-gC-5mer-end-d13 | 682 | 914 or 1092 |
| CTD-gC-5mer-end-d14 | 683 | 915 or 1093 |
| CTD-gC-5mer-end-d15 | 684 | 916 or 1094 |
| CTD-gC-5mer-end-d16 | 685 | 917 or 1095 |
| CTD-gC-5mer-end-d17 | 686 | 918 |
| CTD-gC-5mer-end-d18 | 687 | 919 |
| CTD-gC-5mer-end-d19 | 688 | 920 |
| CTD-gC-5mer-end-d20 | 689 | 921 |
| CTD-gC-5mer-end-d21 | 690 | 922 |
| CTD-gC-5mer-end-d22 | 691 | 923 |
| CTD-gC-5mer-end-d23 | 692 | 924 |
| CTD-gC-5mer-end-d24 | 693 | 925 |
| CTD-gC-5mer-end-d25 | 694 | 926 |
| CTD-gC-5mer-end-d26 | 695 | 927 |
| CTD-gC-5mer-end-d27 | 696 | 928 |
| CTD-gC-5mer-end-d28 | 697 | 929 |
| CTD-gC-5mer-end-d29 | 698 | 930 |
| CTD-gC-5mer-end-d30 | 699 | 931 |
| CTD-gC-5mer-end-d31 | 700 | STN |
| CTD-gC-5mer-end-d32 | 701 | ST |
| CTD-gC-5mer-end-d33 | 702 | S |
| CTD-gC-5mer-end-d34 | 703 | N/A |
| CTD-gC-5mer-end-d35 | 704 | N/A |
| CTD-gC-5mer-end-d36 | 705 | N/A |
| CTD-gC-5mer-end-d37 | 706 | N/A |
| CTD-gC-5mer-end-d38 | 707 | N/A |
| CTD-gC-5mer-end- | 708 | 918 |
| d17_Om | | |
| CTD-gC-5mer-end- | 709 | 919 |
| d18_Om | | |
| CTD-gC-5mer-end- | 710 | 920 |
| d19_Om | | |
| CTD-gC-5mer-end- | 711 | 921 |
| d20_Om | | |
| CTD-gC-5mer-end- | 712 | 922 |
| d21_Om | | |

To be clear, an N-terminus-adjacent region is located at the C-terminal end of an N-terminus region, and a C-terminus-adjacent region is located at the N-terminal end of a C-terminus region. Accordingly, in some embodiments, an immunogenic fragment comprises, consists of, or consists essentially of an amino acid sequence that can be depicted as [an N-terminus region amino acid sequence]-[a corresponding N-terminus-adjacent amino acid sequence]-[an amino acid sequence selected from the group consisting of SEQ ID NOs: 603-606, 2037-2040, and 2045-2050]-[a corresponding C-terminus-adjacent amino acid sequence]-[a C-terminus amino acid sequence], wherein the intervening region comprises, consists of, or consists essentially of the amino acid sequence depicted by the bolded portion. In another embodiment, an immunogenic fragment comprises, consists of, or consists essentially of an amino acid sequence that can be depicted as [an N-terminus region amino acid sequence]-[a corresponding N-terminus-adjacent amino acid sequence]-[an amino acid sequence selected from the group consisting of SEQ ID NOs: 603-606, 2037-2040, and 2045-2050]-[a C-terminus amino acid sequence], wherein the intervening region comprises, consists of, or consists essentially of the amino acid sequence depicted by the bolded portion. In another embodiment, an immunogenic fragment comprises, consists of, or consists essentially of an amino acid sequence that can be depicted as [an N-terminus region amino acid sequence]-[an amino acid sequence selected from the group consisting of SEQ ID NOs: 603-606, 2037-2040, and 2045-2050]-[a corresponding C-terminus-adjacent amino acid sequence]-[a C-terminus amino acid sequence], wherein the intervening region comprises, consists of, or consists essentially of the amino acid sequence depicted by the bolded portion. For instance, an immunogenic fragment can comprise, consist of, or consist essentially of, an amino acid sequence comprising, consisting of, or consisting essentially of an N-terminus region of SEQ ID NO: 627, a corresponding N-terminus-adjacent region of SEQ ID NO: 831, an intervening region of SEQ ID NO: 606, a corresponding C-terminus-adjacent region of SEQ ID NO: 854, and a C-terminus region of SEQ ID NO: 650.

In some embodiments, an immunogenic fragment comprises, consists of, or consists essentially of [an N-terminus amino acid sequence selected from SEQ ID NOs: 611-624, preferably SEQ ID NOs: 611-614, 616-617, 621-627, or 622-624]-[an N-terminus-adjacent amino acid sequence selected from Table 1 that corresponds to the selected N-terminus amino acid sequence]-[an amino acid sequence selected from the group consisting of SEQ ID NOs: 603-606, 2037-2040, and 2045-2050]-[a C-terminus-adjacent amino acid sequence selected from Table 2 that corresponds to the selected N-terminus amino acid sequence]-[a C-terminus amino acid sequence selected from SEQ ID NOs: 688-703 and 710-712, preferably SEQ ID NOs: 688-693, 694-699, 700-703, 691-696, or 697-701], wherein the intervening region comprises, consists of, or consists essentially of the amino acid sequence depicted by the bolded portion.

In some embodiments, there are no amino acid residues between the N-terminus-adjacent region amino acid sequence and the N-terminus region, between the N-terminus-adjacent region amino acid sequence and the amino acid sequence selected from the group consisting of SEQ ID NOs: 603-606, 2037-2040, and 2045-2050, or both, i.e., the sequences directly abut each other.

In some embodiments, there are no additional amino acid residues between the C-terminus-adjacent region amino acid sequence and the C-terminus region, between the C-terminus-adjacent region amino acid sequence and the amino acid sequence selected from the group consisting of SEQ ID NOs: 603-606, 2037-2040, and 2045-2050, or both, i.e., the sequences directly abut each other.

In some embodiments, an immunogenic fragment comprises an N-terminus region and a C-terminus region, wherein the N-terminus region comprises, consists of, or consists essentially of an amino acid sequence of at least five, at least six, at least seven, at least eight, at least nine, at least 10, at least 11, at least 12, at least 13, at least 14, or at least 15 contiguous amino acid residues of an amino acid sequence of SEQ ID NO: 245, 293, 314, or 342. In some embodiments, an immunogenic fragment comprises an N-terminus region and a C-terminus region, wherein the C-terminus region comprises, consists of, or consists essentially of an amino acid sequence of at least five, at least six, at least seven, at least eight, at least nine, at least 10, at least 11, at least 12, at least 13, at least 14, or at least 15 contiguous amino acid residues of an amino acid sequence of SEQ ID NO: 363 or 402.

When an N-terminus region comprises, consists of, or consists essentially of a number of contiguous amino acid residues of an amino acid sequence of SEQ ID NO: 245, 293, 314, or 342, there is at least one corresponding N-terminus-adjacent region amino acid sequence in nearly all cases. Accordingly, in some embodiments, a corresponding N-terminus-adjacent region amino acid sequence can comprise, consist of, or consist essentially of the portion of the amino acid sequence of SEQ ID NO: 245, 293, 314, or 342 that is “downstream” of the contiguous amino acid residues of the N-terminus region, i.e., the portion of the amino acid sequence of SEQ ID NO: 245, 293, 314, or 342 that begins directly after the C-terminal most residue of the contiguous amino acid residues and ends at the C-terminal end of SEQ ID NO: 245, 293, 314, or 342. The only cases in which there is no corresponding N-terminus adjacent region amino acid sequence is when the C-terminal most residue of the contiguous amino acid residues is also the C-terminal most residue of SEQ ID NO: 245, 293, 314, or 342. For example, when the N-terminus region comprises, consists of, or consists essentially of the first 12 contiguous amino acid residues of amino acid sequence SEQ ID NO: 245, then a corresponding N-terminus-adjacent region amino acid sequence comprises, consists of, or consists essentially of all amino acid residues of SEQ ID NO: 245 that are downstream of the 12 contiguous amino acid residues of the N-terminus region.

Similarly, when a C-terminus region comprises, consists of, or consists essentially of such contiguous amino acid residues of an amino acid sequence of SEQ ID NO: 363 or 402, there is at least one corresponding C-terminus-adjacent region amino acid sequence in nearly all cases. Accordingly, in some embodiments, a corresponding C-terminus-adjacent region amino acid sequence can comprise, consist of, or consist essentially of the portion of the amino acid sequence of SEQ ID NO: 363 or 402 that is “upstream” of the contiguous amino acid residues of the C-terminus region, i.e., the portion of the amino acid sequence of SEQ ID NO: 363 or 402 that begins directly before the N-terminal most residue of the contiguous amino acid residues and ends at the N-terminal end of SEQ ID NO: 363 or 402. The only cases in which there is no corresponding C-terminus adjacent region amino acid sequence is when the N-terminal most residue of the contiguous amino acid residues is also the N-terminal most residue of SEQ ID NO: 363 or 402. For example, when the C-terminus region comprises, consists of, or consists essentially of the last 9 contiguous amino acid residues of amino acid sequence SEQ ID NO: 363, then a corresponding C-terminus-adjacent region amino acid sequence comprises, consists of, or consists essentially of all amino acid residues of SEQ ID NO: 363 that are upstream of the 9 contiguous amino acid residues of the C-terminus region.

In some embodiments, an immunogenic fragment further comprises both a corresponding N-terminus-adjacent region, and a corresponding C-terminus-adjacent region, wherein each is as described herein.

In embodiments wherein the intervening region further comprises a corresponding N-terminus-adjacent region, a corresponding C-terminus-adjacent region, or both, the percent identity calculation is based off the amino acid sequence of the entire intervening region amino acid sequence, i.e., the amino acid sequence of the corresponding N-terminal-adjacent region and/or the amino acid sequence of the corresponding C-terminal-adjacent region, combined in the appropriate orientation with the other amino acid sequence(s) of which the intervening region comprises, consists, or consists essentially.

Similarly, in embodiments wherein the intervening region further comprises a corresponding N-terminus-adjacent region, a corresponding C-terminus-adjacent region, or both, the calculation of the number of differing amino acid residues is based off the amino acid sequence of the entire intervening region amino acid sequence, i.e., the amino acid sequence of the N-terminal-adjacent region and/or the amino acid sequence of the C-terminal-adjacent region, combined in the appropriate orientation with the other amino acid sequence(s) of which the intervening region comprises, consists, or consists essentially.

In some embodiments, the intervening region comprises, consists of, consists essentially of an amino acid sequence selected from the group consisting of SEQ ID NOs: 603-606, 2037-2040, and 2045-2050, and optionally a corresponding N-terminus-adjacent region sequence, and/or a corresponding C-terminus-adjacent region sequence, wherein the intervening region amino acid sequence further includes 0 mutations or at least one, at least two, at least three, at least four, at least five, at least six, at least seven, at least eight, at least nine, or at least 10, at least 11, at least 12, at least 13, at least 14, or at least 15 mutations at the following positions of the amino acid sequence, wherein the positions are relative to the first residue of the amino acid sequence selected from the group consisting of SEQ ID NOs: 603-606, 2037-2040, and 2045-2050: −28, −21, 3, 5, 7, 49, 72, 78, 84, 109, 110, 116, 125, 128, 130, 133, 137, and 179. A negative number denotes that the position is upstream (i.e., toward the N-terminal end) of the first residue of the amino acid sequence selected from the group consisting of SEQ ID NOs: 603-606, 2037-2040, and 2045-2050, whereas a positive number denotes that the position is downstream (i.e., toward the C-terminal end) of the first residue.

In some embodiments, the intervening region comprises, consists of, consists essentially of an amino acid sequence selected from the group consisting of SEQ ID NOs: 603-606, 2037-2040, and 2045-2050, and optionally an N-terminus-adjacent region sequence, and/or a C-terminus-adjacent region sequence, wherein the intervening region amino acid sequence further includes no more than one, no more than two, no more than three, no more than four, no more than five, no more than six, no more than seven, no more than eight, no more than nine, or no more than 10, no more than 11, no more than 12, no more than 13, no more than 14, no more than 15, no more than 16, or no more than 17 mutations at the following positions of the amino acid sequence, wherein the positions are relative to the first residue of the amino acid sequence selected from the group consisting of SEQ ID NOs: 603-606, 2037-2040, and 2045-2050: −28, −21, 3, 5, 7, 49, 72, 78, 84, 109, 110, 116, 125, 128, 130, 133, 137, and 179. In further embodiments, the intervening region comprises, consists of, consists essentially of an amino acid sequence selected from the group consisting of SEQ ID NOs: 603-606, 2037-2040, and 2045-2050, and optionally an N-terminus-adjacent region sequence, and/or a C-terminus-adjacent region sequence, wherein the intervening region amino acid sequence further includes a range of the quantity of mutations, wherein the lower limit is any value from the “at least” list in the previous paragraph and the upper limit is any value from the “no more than” list in this paragraph, so long as the lower limit value is less than the upper limit value. For example, the range of the quantity of mutations can be 1-5, 1-4, 1-3, 1-2, 2-5, 2-4, 2-3, 3-5, 3-4, 4-5, 0-5, 0-4, 0-3, 0-2, and 0-1.

In an embodiment, the one or more mutations are L84R and T110K. In another embodiment, the one or more mutations are K49N, L84R, and T110K. In another embodiment, the one or more mutations are R(−)21K, E116K, and N133Y. In another embodiment, the one or more mutations are R(−)21K, T110R, and E116K. In another embodiment, the one or more mutations are R(−)21K, K49N, L84R, 478K, E116K, and N133Y. In another embodiment, the one or more mutations are R(−)21K, K49N, L84R, T110R, E116K, and N133Y. In another embodiment, the one or more mutations are G(−)28D, S3L, S5P, S7F, K49N, N72K, G78S, S109N, T110K, E116A, Q125K, G128S, Q130R, N133Y, and Y137H. In another embodiment, the one or more mutations are G(−)28D, S3L, S5P, S7F, K49N, N72K, G78S, S109N, T110K, E116A, Q125R, G128S, Q130R, N133Y, and Y137H. In another embodiment, the one or more mutations are G(−)28D, R(−)21K, S3L, S5P, S7F, K49N, N72K, G78S, L84R, S109N, T110K, E116K, Q125R, G128S, Q130R, N133Y, and Y137H. In another embodiment, the one or more mutations are G(−)28D, R(−)21K, S3L, S5P, S7F, K49N, N72K, G78S, L84R, S109N, T110K, E116A, Q125R, G128S, Q130R, N133Y, and Y137H. In another embodiment, the one or more mutations are G(−)28D, R(−)21K, S3L, S5P, S7F, K49N, N72K, G78S, L84R, S109N, T110R, E116A, Q125R, G128S, Q130R, N133Y, and Y137H. In another embodiment, the one or more mutations are G(−)28D, R(−)21K, S3L, S5P, S7F, K49N, N72K, G78S, L84R, S109N, T110R, E116K, Q125R, G128S, Q130R, N133Y, and Y137H. In another embodiment, the one or more mutations are G(−)28D, R(−)21K, S3L, S5P, S7F, K49N, N72K, G78S, L84R, S109N, T110R, E116K, Q125R, G128S, Q130R, N133Y, Y137H, and T179K.

In certain embodiments, an immunogenic fragment does not comprise, consist of, or consist essentially of one or more particular amino acid sequences. For instance, in some embodiments, an immunogenic fragment does not comprise, consist of, or consist essentially of an amino sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 171, 173, 175, 177, 179, 181, 183, 185, 187, 189, 191, 199, 201, 203, 205, 207, 209, 211, 213, 215, and 2057-2066, or any combination thereof.

In certain embodiments, a polynucleotide encoding an immunogenic fragment does not comprise, consist of, or consist essentially of one or more particular nucleotide sequences. For instance, in some embodiments, a polynucleotide encoding an immunogenic fragment does not comprise, consist of, or consist essentially of a nucleotide sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 172, 174, 176, 178, 180, 182, 184, 186, 188, 190, 192, 200, 202, 204, 206, 208, 210, 212, 214, 216, and 2067-2076, or any combination thereof.

In some embodiments, an immunogenic fragment does not comprise an intervening region comprising, consisting of, or consisting essentially of an amino acid sequence selected from the group consisting of SEQ ID NOs: 603-606, 2037-2040, and 2045-2050, and optionally an N-terminus-adjacent region sequence, and/or a C-terminus-adjacent region sequence, wherein the intervening region amino acid sequence further comprises one or more of the following amino acid substitutions, wherein the positions are relative to the first residue of the amino acid sequence selected from the group consisting of SEQ ID NOs: 603-606, 2037-2040, and 2045-2050: G(−)28D, R(−)21K, S3L, S5P, S7F, K49N, K49T, N72K, G78S, L84R, S109N, T110K, E116K, E116Q, E116A, Q125R, Q128S, Q130R, N133Y, Y137H, and T179K. In one embodiment, the one or more amino acid substitutions is L84R. In another embodiment, the one or more amino acid substitutions is E116K. In a further embodiment, the one or more amino acid substitutions are K49N, E116K, and N133Y. In yet another embodiment, the one or more amino acid substitutions are K49T, E116K, and N133Y. In another embodiment, the one or more amino acid substitutions are K49T, L84R, E116K, and N133Y. In another embodiment, the one or more amino acid substitutions and/or deletions are L84R; or E116K; or K49N, E116K, and N133Y; or K49T, E116K, and N133Y; or K49T, L84R, E116K, and N133Y.

In some embodiments, an immunogenic fragment comprises, consists of, consists essentially of an amino acid sequence with at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NOs: 1577-1661, and 1917-1946, preferably SEQ ID NOs: 1924, 1925, 1928, 1939, 1940, and 1943. In an embodiment, an immunogenic fragment comprises, consists of, consists essentially of an amino acid sequence at least 95%, at least 96%, at least 97%, at least 98%, or at least 99% identity to an amino acid sequence selected from the group consisting of SEQ ID NOs: 1577-1661, and 1917-1946. In some embodiments, an immunogenic fragment comprises, consists of, or consists essentially of, an amino acid sequence with no more than 70%, no more than 71%, no more than 72%, no more than 73%, no more than 74%, no more than 75%, no more than 76%, no more than 77%, no more than 78%, no more than 79%, no more than 80%, no more than 81%, no more than 82%, no more than 83%, no more than 84%, no more than 85%, no more than 86%, no more than 87%, no more than 88%, no more than 89%, no more than 90%, no more than 91%, no more than 92%, no more than 93%, no more than 94%, no more than 95%, no more than 96%, no more than 97%, no more than 98%, or no more than 99% identity to an amino acid sequence selected from the group consisting of SEQ ID NOs: 1577-1661 and 1917-1946, preferably SEQ ID NOs: 1924, 1925, 1928, 1939, 1940, and 1943. In an embodiment, an immunogenic fragment comprises, consists of, consists essentially of an amino acid sequence no more than 95%, no more than 96%, no more than 97%, no more than 98%, or no more than 99% identity to an amino acid sequence selected from the group consisting of SEQ ID NOs: 1577-1661 and 1917-1946, preferably SEQ ID NOs: 1924, 1925, 1928, 1939, 1940, and 1943.

In some embodiments, an immunogenic fragment comprises, consists of, or consists essentially of, an amino acid sequence with a range of percent identity to an amino acid sequence selected from the group consisting of SEQ ID NOs: 1577-1661 and 1917-1946, preferably SEQ ID NOs: 1924, 1925, 1928, 1939, 1940, and 1943, wherein the percent identity value of the bottom of the range is any value from the “at least” list of values recited in the previous paragraph, e.g., 87%, and the percent identity value for the top of the range is any value from “no more than” list recited in the previous paragraph, e.g., 96%, as long as the bottom range value is less than the top range value. For example, a range of percent identity can be between 87% and 94% identity. In some embodiments, the range of percent identity is 90-99, 90-98, 90-97, 90-96, 90-95, 90-94, 90-93, 90-92, 90-91, 91-99, 91-98, 91-97, 91-96, 91-95, 91-94, 91-93, 91-92, 92-99, 92-98, 92-97, 92-96, 92-95, 92-94, 92-93, 93-99, 93-98, 93-97, 93-96, 93-95, 93-94, 94-99, 94-98, 94-97, 94-96, 94-95, 95-99, 95-98, 95-97, 95-96, 96-99, 96-98, 96-97, 97-99, 97-98, or 98-99.

In some embodiments, an immunogenic fragment comprises, consists of, or consists essentially of, an amino acid sequence that differs at 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues from the amino acid sequence of an amino acid sequence selected from the group consisting of SEQ ID NOs: 1577-1661 and 1917-1946, preferably SEQ ID NOs: 1924, 1925, 1928, 1939, 1940, and 1943. In some embodiments, the number of differing amino acid residues is 0, 1, 2, 3, 4, 5, 6, 7, 8, 9, 10, 11, 12, 13, 14, 15, 16, 17, 18, 19, or 20.

In some embodiments, an immunogenic fragment comprises, consists of, or consists essentially of, an amino acid sequence that differs at no residues or at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 residues from the amino acid sequence of an amino acid sequence selected from the group consisting of SEQ ID NOs: 1577-1661 and 1917-1946, preferably SEQ ID NOs: 1924, 1925, 1928, 1939, 1940, and 1943. In some embodiments, the number of differing amino acid residues is 0, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20. In some embodiments, the immunogenic fragment comprises, consists of, or consists essentially of, an amino acid sequence that differs from an amino acid sequence selected from the group consisting of SEQ ID NOs: 1577-1661 and 1917-1946, preferably SEQ ID NOs: 1924, 1925, 1928, 1939, 1940, and 1943, at no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 21, no more than 22, no more than 23, no more than 24, no more than 25, no more than 26, no more than 27, no more than 28, no more than 29, no more than 30, no more than 31, no more than 32, no more than 33, no more than 34, no more than 35, no more than 36, no more than 37, no more than 38, no more than 39, no more than 40, no more than 41, no more than 42, no more than 43, no more than 44, no more than 45, no more than 46, no more than 47, no more than 48, no more than 49, or no more than 50 residues. In some embodiments, the number of differing amino acid residues is no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, or no more than 20.

In some embodiments, the immunogenic fragment comprises, consists of, or consists essentially of, an amino acid sequence with a range of residues at which the sequence differs from an amino acid sequence selected from the group consisting of SEQ ID NOs: 1577-1661 and 1917-1946, preferably SEQ ID NOs: 1924, 1925, 1928, 1939, 1940, and 1943, wherein the minimum number of differing residues is any value from the “at least” list recited in the previous paragraph, e.g., 4 residues, and the maximum number of differing residues is any value from the “no more than” list recited in the previous paragraph, e.g., 33 residues, as long as the bottom range value is less than the top range value. For example, a range of differing residues can be between 3 and 9 residues. In some embodiments, the range of differing residues is 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, −5-9, 5-8, 5-7, 5-6, 6-10, 6-9, 6-8, 6-7, 7-10, 7-9, 7-8, 8-10, 8-9, 9-10, 0-10, 0-1, 0-2, 0-3, 0-4, 0-5, 0-6, 0-7, 0-8, 0-9, or 0-10.

Without being bound to theory, the fragments of the SARS-CoV-2 glycoprotein described herein are each considered to be immunogenic, although the magnitude of the immunogenic response can vary upon a number of factors. Immunogenicity can be measured by any suitable method, including, but not limited to, vaccination of a test subject with a suitable dosage of a vaccine composition containing one or more immunogenic fragments, followed by analysis of antibody titers after certain periods of time, e.g., after 14 days, and/or after 28 days. Antibody analysis can be by any suitable method, including ELISA analysis. Test subjects can be any suitable subject, including mammals, such as primates, e.g., rhesus macaques, camelids (e.g., camel, llama, alpaca, dromedary and guanaco), rodents, e.g., mice and rats, and humans. Dosage can be any suitable dosage, including 12.5 ug, 25 ug, or 50 ug. Factors that influence immunogenic response include, but are not limited to, other components of the vaccine composition, e.g., adjuvants, the dosage and/or frequency at which the vaccine composition is administered, the type of subject, and the period of time between administration of the vaccine composition and subsequent measurement of the antibody titer.

In some embodiments, the immunogenic fragment is encoded by a recombinant polynucleotide. The recombinant polynucleotide can be any suitable polynucleotide. In some embodiments, the nucleotide sequence of the polynucleotide is codon optimized and/or codon pair optimized. In some embodiments, the recombinant polynucleotide encoding an immunogenic fragment is encoded by a nucleotide sequence that comprises, consists of, or consists essentially of a nucleotide sequence with at least 90%, preferably at least 91%, 92%, 93%, 94%, 95%, 96%, 97%, 98%, or 99%, sequence identity to a nucleotide sequence selected from the group consisting of nucleotide sequence selected from the group consisting of SEQ ID NOs: SEQ ID NOs: 1747-1831 and 1977-2006. In some embodiments, the recombinant polynucleotide encoding an immunogenic fragment is encoded by a nucleotide sequence that comprises, consists of, or consists essentially of a nucleotide sequence selected from the group consisting of SEQ ID NOs: 1747-1831 and 1977-2006.

Nucleotide or amino acid sequence “identity,” as referenced herein, can be determined by comparing a nucleotide or amino acid sequence of interest to a reference nucleotide or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100×[(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

Calculation of the number of residues at which two amino acid or nucleotide sequences differ, as referenced herein, can be determined in a similar fashion, i.e., by comparing a nucleotide or amino acid sequence of interest to a reference nucleotide or amino acid sequence. In contrast to the calculation of percent identity, in which the number of nucleotides or amino acid residues that are the same (i.e., that are identical) as between the optimally aligned sequence of interest and the reference sequence is divided by the length of the longest sequence (i.e., the length of either the sequence of interest or the reference sequence, whichever is longer), the calculation is simply summation of the number of nucleotides or amino acid residues that are not the same (i.e., that are not identical) as between the optimally aligned sequence of interest and the reference sequence. As before, alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

### Description of Polypeptides Including at Least One Immunogenic Fragment

Polypeptides of the invention include any suitable immunogenic fragment or combination of such fragments, such as those described herein. In some embodiments, the polypeptides are recombinant and/or isolated. Accordingly, recombinant polypeptides of the invention include at least one immunogenic fragment. Unless otherwise indicated, each immunogenic fragment embodiment described herein can be combined together in any order and in any reasonable quantity within a recombinant polypeptide. In some embodiments, the recombinant polypeptide comprises, consists of, or consist essentially of at least one, at least two, at least three, at least four, at least five, at least six, at least seven, at least eight, at least nine, at least 10, at least 11, at least 12, at least 13, at least 14 or at least 15 immunogenic fragments. In some embodiments, the recombinant polypeptide comprises, consists of, or consist essentially of, no more than one, no more than two, no more than three, no more than four, no more than five, no more than six, no more than seven, no more than eight, no more than nine, no more than 10, no more than 11, no more than 12, no more than 13, no more than 14 or no more than 15 immunogenic fragments.

In some embodiments, the recombinant polypeptide comprises, consists of, or consist essentially of a range of quantity of immunogenic fragments, wherein the minimum number of immunogenic fragments is any value from the “at least” list recited in the previous paragraph, e.g., 4 immunogenic fragments, and the maximum number of immunogenic fragments is any value from the “no more than” list recited in the previous paragraph, e.g., 6 immunogenic fragments, as long as the bottom range value is less than the top range value. For example, a range of immunogenic fragments can be between 1 and 4 immunogenic fragments. In some embodiments, a recombinant polypeptide comprises, consists of, or consist essentially of 1-5, 1-4, 1-3, 1-2, 2-5, 2-4, 2-3, 3-5, 3-4, or 4-5 immunogenic fragments.

In some embodiments, a recombinant polypeptide comprises, consists of, or consists essentially of, one, two, three, four, five, six, seven, eight, nine, ten, eleven, twelve, thirteen, fourteen, or fifteen immunogenic fragments. In an embodiment, a recombinant polypeptide comprises, consists of, or consists essentially of one, two, or three immunogenic fragments. In an embodiment, a recombinant polypeptide comprises, consists of, or consists essentially of one or two immunogenic fragments.

(In some embodiments, the recombinant polypeptide comprises, consists of, or consists essentially of, a plurality of identical immunogenic fragments. For example, a recombinant polypeptide can include two, three, four, five, six, seven, eight, nine, ten, or more immunogenic fragments, wherein each fragment comprises, consists of, or consists essentially of the same amino acid sequence. In some embodiments, the recombinant polypeptide includes two, three, four or five identical immunogenic fragments. In yet further embodiments, the recombinant polypeptide comprises, consists of, or consists essentially of two or three identical immunogenic fragments. In another embodiment, the recombinant polypeptide comprises, consists of, or consists essentially of two identical immunogenic fragments.

In some embodiments, the recombinant polypeptide includes a plurality of non-identical immunogenic fragments. For example, a recombinant polypeptide can include two, three, four, five, six, seven, eight, nine, ten, or more immunogenic fragments, wherein each fragment comprises, consists of, or consists essentially of a different amino acid sequence from each other fragment, i.e., each fragment is a different fragment. In some embodiments, the recombinant polypeptide comprises, consists of, or consists essentially of two, three, four or five different immunogenic fragments. In yet another embodiment, the recombinant polypeptide comprises, consists of, or consists essentially of two or three different immunogenic fragments. In another embodiment, the recombinant polypeptide comprises, consists of, or consists essentially of two different immunogenic fragments.

In some embodiments, the recombinant polypeptide includes a plurality of immunogenic fragments, in which some of the fragments are identical, but not all. For example, a recombinant polypeptide can include two, three, four, five, six, seven, eight, nine, ten, or more immunogenic fragments, wherein each fragment comprises, consists of, or consists essentially of the same amino acid sequence, while also including one, two, three, four, five, six, seven, eight, nine, ten, or more immunogenic fragments, wherein each fragment comprises, consists of, or consists essentially of a different amino acid sequence from each other fragment. In some embodiments, the recombinant polypeptide comprises, consists of, or consists essentially of a total of two, three, four or five immunogenic fragments. In yet further embodiments, the recombinant polypeptide comprises, consists of, or consists essentially of a total of two or three immunogenic fragments, wherein at least one immunogenic fragment is different from the other immunogenic fragments within the polypeptide.

In some embodiments, the recombinant polypeptide comprises, consists of, or consists essentially of at least two immunogenic fragments of SARS-CoV-2 spike glycoprotein, wherein the at least two immunogenic fragments each comprise an N-terminus region, wherein the N-terminus region of each fragment comprises an amino acid sequence independently selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355. In some embodiments, the recombinant polypeptide comprises, consists of, or consists essentially of at least two immunogenic fragments of SARS-CoV-2 spike glycoprotein, wherein the at least two immunogenic fragments each comprise N-terminus and C-terminus regions, wherein the N-terminus region of each fragment comprises an amino acid sequence independently selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and wherein the C-terminus region comprises, consists of, or consists essentially of an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423. In some embodiments, the recombinant polypeptide comprises, consists of, or consists essentially of at least two immunogenic fragments of SARS-CoV-2 spike glycoprotein, wherein the at least two immunogenic fragments each comprise an N-terminus region and a C-terminus region, wherein the N-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and wherein the C-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423. In some embodiments, the recombinant polypeptide comprises, consists of, or consists essentially of at least two immunogenic fragments of SARS-CoV-2 spike glycoprotein, wherein the at least two immunogenic fragments each comprise an N-terminus region, an intervening region, and a C-terminus region, wherein the N-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and wherein the C-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423, and wherein the intervening region comprises, consists of, or consists essentially of, an amino acid sequence that differs at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues from an amino acid sequence selected from the group consisting of SEQ ID NOs: 603-606, 2037-2040, and 2045-2050.

In some embodiments, the recombinant polypeptide comprises, consists of, or consists essentially of at least two immunogenic fragments of SARS-CoV-2 spike glycoprotein, wherein the at least two immunogenic fragments each comprise an N-terminus region, an intervening region, and a C-terminus region, wherein the N-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and wherein the C-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 363-398 and 402-423, and amino acid sequences STN, ST, and S, preferably selected from SEQ ID NOs:382-397, 382-387, 388-393, 394-397, 385-390, 391-395, and 421-423, and wherein the intervening region comprises, consists of, or consists essentially of, an amino acid sequence with at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 603-606, 2037-2040, and 2045-2050.

In some embodiments, the recombinant polypeptide comprises, consists of, or consists essentially of at least three immunogenic fragment of SARS-CoV-2 spike glycoprotein, wherein the at least three immunogenic fragments each comprise an N-terminus region, an intervening region, and a C-terminus region, wherein the N-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and wherein the C-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423, and wherein the intervening region comprises, consists of, or consists essentially of, an amino acid sequence with at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 603-606, 2037-2040, and 2045-2050.

In some embodiments, the recombinant polypeptide comprises, consists of, or consists essentially of at least three immunogenic fragment of SARS-CoV-2 spike glycoprotein, wherein the at least three immunogenic fragments each comprise an N-terminus region, an intervening region, and a C-terminus region, wherein the N-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and wherein the C-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423, and wherein the intervening region comprises, consists of, or consists essentially of, an amino acid sequence that differs at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues from an amino acid sequence selected from the group consisting of SEQ ID NOs: 603-606, 2037-2040, and 2045-2050.

In some embodiments, the recombinant polypeptide comprises, consists of, or consists essentially of two immunogenic fragment of SARS-CoV-2 spike glycoprotein, wherein the two immunogenic fragments each comprise an N-terminus region, an intervening region, and a C-terminus region, wherein the N-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and wherein the C-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423, and wherein the intervening region comprises, consists of, or consists essentially of, an amino acid sequence with at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 603-606, 2037-2040, and 2045-2050.

In some embodiments, the recombinant polypeptide comprises, consists of, or consists essentially of two immunogenic fragment of SARS-CoV-2 spike glycoprotein, wherein the two immunogenic fragments each comprise an N-terminus region, an intervening region, and a C-terminus region, wherein the N-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and wherein the C-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423, and wherein the intervening region comprises, consists of, or consists essentially of, an amino acid sequence that differs at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues from an amino acid sequence selected from the group consisting of SEQ ID NOs: 603-606, 2037-2040, and 2045-2050.

In some embodiments, the recombinant polypeptide comprises, consists of, or consists essentially of three immunogenic fragment of SARS-CoV-2 spike glycoprotein, wherein the three immunogenic fragments each comprise an N-terminus region, an intervening region, and a C-terminus region, wherein the N-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and wherein the C-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423, and wherein the intervening region comprises, consists of, or consists essentially of, an amino acid sequence with at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO: 603-606, 2037-2040, and 2045-2050.

Recombinant polypeptides of the invention can comprise, consist of, or consist essentially of any combination of immunogenic fragments disclosed herein. For example, in one embodiment, when a recombinant polypeptide comprises, consists of, or consists essentially of three immunogenic fragments, wherein the at least three immunogenic fragments each comprise an N-terminus region, an intervening region, and a C-terminus region, wherein the N-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and wherein the C-terminus region of each immunogenic fragment comprises, consists of, or consists essentially of an amino acid sequence that differs at a total of 1, 2, 3, 4, 5, 6, 7, 8, 9, 10, 11, 12, 13, 14, or 15 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423, and wherein the intervening region comprises, consists of, or consists essentially of, an amino acid sequence that differs at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues from an amino acid sequence selected from the group consisting of SEQ ID NOs: 603-606, 2037-2040, and 2045-2050.

In some embodiments, wherein the recombinant polypeptide includes a plurality of immunogenic fragments, the immunogenic fragments are connected to each other via a linker. The linker can be any suitable linker. Suitable linkers include a polypeptide comprising, consisting of, or consisting essentially of, an amino acid sequence of 1-35 residues, wherein each residue is independently serine, glycine, or aspartic acid, and further wherein the amino acid sequence contains zero or one aspartic acid residues. Other suitable linkers include a polypeptide comprising an amino acid sequence selected from the group consisting of SEQ ID NO: 65 (Fc1), SEQ ID NO: 67 (Fc1-TEV), SEQ ID NO: 69 (Fc1-Rv3C), SEQ ID NO: 193 (Short), SEQ ID NO: 195 (Medium), and SEQ ID NO: 197 (Long). When a recombinant polypeptide includes two or more such linkers, the amino acid sequence of the linkers can be identical or different. In some embodiments, wherein the recombinant polypeptide includes a plurality of immunogenic fragments, the immunogenic fragments are connected directly to each other without an intervening linker.

Certain embodiments of the recombinant polypeptide that include a plurality of immunogenic fragments provide for, for instance, a flexible expression platform with robust expression of full-length protein, modality to modify individual or multiple domains within one or more of the plurality of immunogenic fragments to reflect the most recent virus variant sequence(s), allow for single step affinity purification, in certain embodiments achieving high levels of purity, e.g., greater than 90%), and provide high-level, long-term immune response as tested in Rhesus macaques.

The inventors have found that recombinant polypeptides of the invention containing immunogenic fragments comprising certain endpoint sequences exhibit one or more unexpected and/or surprising properties. Such surprising properties of the recombinant polypeptides include, but are not limited to, unusually high levels of protein expression, unusually high levels of protease resistance, and unusually high resistance to protein precipitation in solution. These properties are all of particular relevance to the production, use and effectiveness of biologically active and economically viable vaccine compositions.

In some embodiments, the recombinant polypeptide of the invention can be expressed at a concentration of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, or at least 130 mg of protein per liter of culture (e.g., “mg protein/L culture”). In some embodiments, the concentration is a range, wherein the lower and upper limit of the range are values from the preceding “at least” list, so long as the lower limit value is less than the upper limit value. For instance, a range can be 50-120 mg protein/L culture. In an embodiment, the concentration is at least 5 mg protein/L culture. In another embodiment, the concentration is at least 30 mg protein/L culture. In another embodiment, the concentration is at least 100 mg protein/L culture.

Quantitation of protein concentration can be by any suitable method. For instance, protein concentration can be calculated using absorbance of 280 nm light and protein-specific extinction coefficients determined in silico according to the expected amino acid composition of the recombinant polypeptide. Protein-specific extinction coefficients can be determined by any suitable method in the art, including the ExPaSy ProtParam tool at web.expasy.org/protparam (Gasteiger et al., Protein Identification and Analysis Tools on the ExPASy Server; (In) John M. Walker (ed): The Proteomics Protocols Handbook, Humana Press (2005). pp. 571-607) or the protein characterization tools provided within the SNAPGENE™ viewer software platform.

Moreover, an embodiment of the invention is a method of producing a vaccine composition, wherein the method comprises, consists of, or consists essentially of culturing a recombinant polynucleotide of the invention as described herein, whereby a recombinant polypeptide as described herein is produced, wherein the culturing process yields a concentration of the recombinant polypeptide of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, or at least 130 mg of protein per liter of culture (e.g., “mg protein/L culture”). In some embodiments, the concentration is a range, wherein the lower and upper limit of the range are values from the preceding “at least” list, so long as the lower limit value is less than the upper limit value. For instance, a range can be 50-120 mg protein/L culture. In an embodiment, the concentration is at least 5 mg protein/L culture. In another embodiment, the concentration is at least 30 mg protein/L culture. In another embodiment, the concentration is at least 100 mg protein/L culture. In one embodiment, the vaccine production method includes only a single affinity chromatography step for purification to yield a pharmaceutically pure vaccine composition, e.g., Protein A-resin or Protein G resin. In this embodiment, no additional binding of antigenic protein followed by elution is used to further purify the vaccine composition.

The culture or fermentation process for achieving such concentrations of the recombinant polypeptide can be any suitable culture or fermentation process. In some embodiments, the process is carried out in CHO cells and utilizes a defined media kit, e.g., MIRUS™ CHO-S. In certain embodiments, the recombinant polypeptide is encoded by a recombinant polynucleotide, wherein the recombinant polynucleotide is a vector selected from the group consisting of a pcDNA3.1, a pSV, a pCMV, a pBApo-CMV, and a pBApo-EFlalpha expression vector, preferably a pcDNA3.1 vector. In certain embodiments, the vector is transiently transfected into CHO-S cells, wherein the recombinant polypeptide encoded by the vector is expressed under the control of a constitutive promoter within cell culture conditions ranging from 30-37° C. and 1-14 days in CO₂ (8%) incubators with rotary shaking agitation at speeds of 150 RPM.

In some embodiments, the recombinant polypeptides exhibit unusually high protease resistance during purification. Purification can be by any suitable purification process, for example by affinity purification, such as protein-A agarose single step purification. In some instances, such purification is desirable due to its highly scalable nature. Protease resistance can be analyzed using any suitable method, for example by SDS-PAGE analysis. For example, protease degradation during protein production in the cell can be determined using SDS-PAGE and protein staining after affinity purification with Protein-A resin. Following such purification, a recombinant polypeptide can be subjected to SDS-PAGE electrophoresis. Protease degraded protein can be observed as protein bands within a sample that are smaller than the expected full-length proteins. This can be quantified as a percentage of total protein using densitometry analysis features contained within the image analysis software such as ImageJ (Image Processing and Analysis in Java; from NIH; (imagej.nih.gov/ij/index.html) or other suitable software. The densitometry values of all major protein species are summed and then used as a denominator to calculate percentages of individual proteins. A protein resistance percentage can thus be calculated by dividing the densitometry value of the full-length protein by the sum of all identified major protein species. Once a base-line percentage of degraded protein is set at the time of purification for a given lot, protease degradation during storage and/or subsequent handling can be assessed using similar methods to determine if the percentage degradation has shifted. Mass spectrometry can also be used to positively identify and characterize the proteins and/or breakdown products of interest (e.g., degradation products, sites of proteolysis, etc, or whether there are host cell proteins contained within the preparation).

In some embodiments, the recombinant polypeptide exhibits at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% protease resistance. In an embodiment, the recombinant polypeptide exhibits at least 90% protease resistance. In an embodiment, the recombinant polypeptide exhibits at least 91% protease resistance. In an embodiment, the recombinant polypeptide exhibits at least 92% protease resistance. In an embodiment, the recombinant polypeptide exhibits at least 93% protease resistance. In an embodiment, the recombinant polypeptide exhibits at least 94% protease resistance. In an embodiment, the recombinant polypeptide exhibits at least 95% protease resistance. In an embodiment, the recombinant polypeptide exhibits at least 96% protease resistance. In an embodiment, the recombinant polypeptide exhibits at least 97% protease resistance. In an embodiment, the recombinant polypeptide exhibits at least 98% protease resistance. In an embodiment, the recombinant polypeptide exhibits at least 99% protease resistance.

In some embodiments, the recombinant polypeptides exhibit unusually high resistance to protein precipitation in solution. The amount of protein precipitation in a solution is indicative of the amount of misfolded or aggregated protein contained within the solution. The amount of protein precipitation can be measured using any suitable method in the art. For instance, one method (“heat treatment method”) includes heating a protein-containing solution from a baseline temperature to a specific higher temperature, allowing the protein-containing solution to remain at that specific higher temperature for a specific amount of time, allowing the protein containing solution to return to room temperature, and then assaying the protein-containing solution. Suitable assays include visual assessment and scoring of turbidity, measurement of precipitate by weight following centrifugation, SDS-PAGE electrophoresis, mass spectrometry, and UV-Visible spectrometry. When visual assessment and scoring of turbidity is used, a scoring system from 0, meaning totally transparent, no precipitated material, to + to ++ to +++ to ++++, is employed. In some embodiments, using such a scoring system, recombinant polypeptide-containing solutions scored a 0 following the heat treatment method described above. Alternatively, a spectrophotometric determination may be made by light scattering of the protein-containing solution of 600 nanometer light on a UV-visible spectrophotometer can be used to determine the extent of precipitation or aggregation of the protein sample. In general, lower absorbances of 600 nanometers corresponds to lower precipitation or aggregation of protein from solution. Ranges of of Absorbance (light scattering) of 600 nanometer light can be divided into ranges of light scattering from low to no scattering caused from precipitation or aggregation, for example; 0.000 to 0.050 A600 (absorbance of 600 nm light), low precipitation or aggregation could exhibit scattering of 0.050-0.200 A600, medium precipitation or aggregation could exhibit scattering of 0.200 to 0.500 A600, high precipitation or aggregation can exhibit scattering of 0.500 to 1.000 A600, exceptionally high precipitation or aggregation can exhibit scattering of 1.000 A600 or greater turbidity. Accordingly, in some embodiments, a recombinant polypeptide exhibits 0.000 to 0.200, 0.050-0.200, or 0.000 to 0.050 A600 absorbance.

In certain embodiments, a recombinant polypeptide does not comprise, consist of, or consist essentially of one or more particular amino acid sequences. For instance, in some embodiments, a recombinant polypeptide does not comprise, consist of, or consist essentially of an amino sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 199, 201, 203, 205, 207, 209, 211, 213, 215, 217, 219, 221, 223, 225, 227, 229, 231, 233, 235, 237, 239, 241, 243, and 2057-2066, or any combination thereof. In another embodiment, a recombinant polypeptide does not comprise, consist of, or consist essentially of an amino sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 217, 219, 221, 223, 225, 227, 229, 231, 233, 235, 237, 239, 241, 243, and 2057-2066, or any combination thereof.

In certain embodiments, a polynucleotide encoding a recombinant polypeptide does not comprise, consist of, or consist essentially of one or more particular nucleotide sequences. For instance, in some embodiments, a polynucleotide encoding an immunogenic fragment does not comprise, consist of, or consist essentially of a nucleotide sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 200, 202, 204, 206, 208, 210, 212, 214, 216, 218, 220, 222, 224, 226, 228, 230, 232, 234, 236, 238, 240, 242, 244, and 2067-2076, or any combination thereof. In another embodiment, a polynucleotide encoding an immunogenic fragment does not comprise, consist of, or consist essentially of a nucleotide sequence selected from the group consisting of 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 218, 220, 222, 224, 226, 228, 230, 232, 234, 236, 238, 240, 242, 244, and 2067-2076, or any combination thereof.

In some embodiments, a recombinant polypeptide does not comprise, consist of, or consist essentially of an immunogenic fragment comprising an intervening region comprising, consisting of, or consisting essentially of an amino acid sequence selected from the group consisting of SEQ ID NOs: 603-606, 2037-2040, and 2045-2050, and optionally an N-terminus-adjacent region sequence, and/or a C-terminus-adjacent region sequence, wherein the intervening region amino acid sequence further comprises one or more of the following amino acid substitutions, wherein the positions are relative to the first residue of the amino acid sequence selected from the group consisting of SEQ ID NOs: 603-606, 2037-2040, and 2045-2050: G(−)28D, R(−)21K, S3L, S5P, S7F, K49N, K49T, N72K, G78S, L84R, S109N, T110K, E116K, E116Q, E116A, Q125R, Q128S, Q130R, N133Y, Y137H, and T179K. In one embodiment, the one or more amino acid substitutions is L84R. In another embodiment, the one or more amino acid substitutions is E116K. In a further embodiment, the one or more amino acid substitutions are K49N, E116K, and N133Y. In yet another embodiment, the one or more amino acid substitutions are K49T, E116K, and N133Y. In another embodiment, the one or more amino acid substitutions are K49T, L84R, E116K, and N133Y. In another embodiment, the one or more amino acid substitutions and/or deletions are L84R; or E116K; or K49N, E116K, and N133Y; or K49T, E116K, and N133Y; or K49T, L84R, E116K, and N133Y.

In some embodiments, the recombinant polypeptide further comprises, consists of, or consists essentially of an amino acid sequence that encodes an antibody Fc region. The antibody Fc region can be any suitable antibody Fc region. Suitable antibody Fc regions include wild-type human or other animal Immunoglobulin Fc regions such as IgG, IgA, IgD, IgE, IgM, and their respective subclasses, preferably IgA and its respective subclasses, and for example, human IgG1 Fc regions and Fc regions derived therefrom. Other suitable antibody Fc regions include mutant Fc regions that enhance or diminish Fc-receptor binding affinity to speed up or slow down uptake, respectively. In some embodiments, the antibody Fc region comprises the amino acid sequence of SEQ ID NO: 71.

In some embodiments containing an antibody Fc region, the at least one immunogenic fragment are connected to the antibody Fc region via a linker. The linker can be any suitable linker. Suitable linkers include a polypeptide comprising an amino acid sequence of 1-35 residues, wherein each residue is independently serine, glycine, or aspartic acid, and further wherein the amino acid sequence contains zero or one aspartic acid residues. Other suitable linkers include a polypeptide comprising an amino acid sequence selected from the group consisting of SEQ ID NO: 65 (Fc1), SEQ ID NO: 67 (Fc1-TEV), SEQ ID NO: 69 (Fc1-Rv3C), SEQ ID NO: 193 (Short), SEQ ID NO: 195 (Medium), and SEQ ID NO: 197 (Long). When a recombinant polypeptide includes two or more such linkers, the amino acid sequence of the linkers can be identical or different from each other. In some embodiments, wherein the recombinant polypeptide includes at least one immunogenic fragment connected to the N-terminus of the Fc region, and/or at least one immunogenic fragment is connected to the C-terminus of the Fc region, the immunogenic fragment(s) nearest to the Fc region are connected directly to the Fc region without an intervening linker.

Within the recombinant polypeptide, the at least one immunogenic fragment can be arranged in any suitable serial orientation with respect to the Fc region. In some embodiments, the at least one immunogenic fragment is connected to the N-terminus of the Fc region. This orientation can be depicted as [immunogenic fragment]x—[N-terminus—Fc region—C-terminus], wherein X is an integer 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, −5-9, 5-8, 5-7, 5-6, 6-10, 6-9, 6-8, 6-7, 7-10, 7-9, 7-8, 8-10, 8-9, or 9-10 representing the number of immunogenic fragments within the recombinant polypeptide. In some embodiments, the at least one immunogenic fragment is connected to the C-terminus of the Fc region. This orientation can be depicted as [N-terminus—Fc region—C-terminus]—[immunogenic fragment]x, wherein X is an integer 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, −5-9, 5-8, 5-7, 5-6, 6-10, 6-9, 6-8, 6-7, 7-10, 7-9, 7-8, 8-10, 8-9, or 9-10 representing the number of immunogenic fragments within the recombinant polypeptide. In some embodiments, wherein the recombinant polypeptide includes at least two immunogenic fragments, at least one immunogenic fragment is connected to the N-terminus of the Fc region, and at least one immunogenic fragment is connected to the C-terminus of the Fc region. This orientation can be depicted as [immunogenic fragment]x—[N-terminus—Fc region—C-terminus]—[immunogenic fragment]Y, wherein X and Y are independently an integer 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, −5-9, 5-8, 5-7, 5-6, 6-10, 6-9, 6-8, 6-7, 7-10, 7-9, 7-8, 8-10, 8-9, or 9-10 representing the number of immunogenic fragments connected to each side of the Fc region. In some embodiments, wherein the recombinant polypeptide includes two immunogenic fragments, both immunogenic fragments are connected to the N-terminus of the Fc region. This orientation can be depicted as [immunogenic fragment]—[immunogenic fragment]—[N-terminus—Fc region—C-terminus]. In other embodiments, wherein the recombinant polypeptide includes two immunogenic fragments, both immunogenic fragments are connected to the C-terminus of the Fc region. This orientation can be depicted as [N-terminus—Fc region—C-terminus]—[immunogenic fragment][immunogenic fragment]. In some embodiments, wherein the recombinant polypeptide includes three immunogenic fragments, each immunogenic fragment is connected to the N-terminus of the Fc region. This orientation can be depicted as [immunogenic fragment]-[immunogenic fragment]—[immunogenic fragment]—[N-terminus—Fc region—C-terminus]. In other embodiments, wherein the recombinant polypeptide includes three immunogenic fragments, each immunogenic fragment is connected to the C-terminus of the Fc region. This orientation can be depicted as [N-terminus—Fc region—C-terminus]—[immunogenic fragment]—[immunogenic fragment]—[immunogenic fragment].

In some embodiments, the recombinant polypeptide comprises, consists of, or consists essentially of, an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NOs: 1662-1746 and 1947-1976. In some embodiments, the recombinant polypeptide comprises, consists of, or consists essentially of, an amino acid sequence selected from the group consisting of SEQ ID NOs: 1662-1746 and 1947-1976. In some embodiments, the recombinant polypeptide comprises, consists of, or consists essentially of, an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NOs: 1954, 1955, 1958, 1969, 1970, and 1973. In some embodiments, the recombinant polypeptide comprises, consists of, or consists essentially of, an amino acid sequence selected from the group consisting of SEQ ID NOs: 1954, 1955, 1958, 1969, 1970, and 1973.

In some embodiments, the recombinant polypeptide is encoded by a recombinant polynucleotide. In certain embodiments, the polynucleotide comprises, consists of, or consists essentially of a nucleic acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a nucleotide sequence selected from the group consisting of SEQ ID NOs: 1832-1916 and 2007-2036. In some embodiments, the polynucleotide comprises, consists of, or consists essentially of a nucleic acid sequence selected from the group consisting of SEQ ID NOs: 1832-1916 and 2007-2036. In some embodiments, the recombinant polypeptide is encoded by a recombinant polynucleotide. In certain embodiments, the polynucleotide comprises, consists of, or consists essentially of a nucleic acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a nucleotide sequence selected from the group consisting of SEQ ID NOs: 2014, 2015, 2018, 2029, 2030, and 2033. In some embodiments, the polynucleotide comprises, consists of, or consists essentially of a nucleic acid sequence selected from the group consisting of SEQ ID NOs: 2014, 2015, 2018, 2029, 2030, and 2033.

In some cases it is desirable to prepare a recombinant polypeptide suitable as a vaccine which does not comprise an Fc-region. However, the presence of an Fc region in a polypeptide can be, in certain cases, advantageous, insofar as the Fc region can allow for streamlined purification of the recombinant polypeptide and can increase the stability of the recombinant polypeptide. Accordingly, in some embodiments, a recombinant polypeptide comprising an Fc region is modified, such that the Fc region is removed. In some embodiments, the Fc region is removed enzymatically by any method suitable in the art. Non-limiting examples of suitable enzymes include papain, pepsin and IdeS. When a single recombinant polypeptide comprising an Fc region is so enzymatically treated, the result is two fragments, an Fc region-containing fragment and a non-Fc-containing fragment, which comprises the at least one immunogenic fragment.

In some embodiments, two recombinant polypeptides each comprising an Fc region form a dimer, wherein the Fc region of the first polypeptide associates with the Fc region of a second polypeptide (e.g., by means of disulfide bonds). When the dimer is enzymatically treated to remove the Fc region, the result varies depending on the enzyme used. In the case of papain, for example, the dimer is cleaved N-terminal to bridging disulfides in the Fc region producing 3 separate fragments: two Fab fragments and an Fc fragment. In the case of pepsin or IdeS, for example, the dimer is cleaved at the Fc region C-terminal to the bridging disulfide bonds, thereby creating a F(ab′)2, i.e., one fragment lacking the Fc region but still containing the at least one immunogenic fragment of each recombinant polypeptide, and a separate Fc region-containing polypeptide. Alternatively or in addition, protease cleavage sites can be encoded in the recombinant polynucleotide sequence encoding the recombinant polypeptide to produce a cleavable polypeptide using an array of proteases, including but not limited to: TEV protease, factor Xa protease, 2A protease, and 3C protease. Following digestion, the resultant recombinant polypeptide can be purified away from Fc polypeptides (or incompletely digested starting material) by Protein A or protein G subtraction chromatography, leaving a purified recombinant polypeptide free of Fc protein or Fc-containing protein, e.g., an Fc-free protein.

In some embodiments, the recombinant polypeptide includes at least three immunogenic fragments, of which at least one is a SARS-CoV-2 spike glycoprotein fragment as described herein, at least one is a SARS-CoV spike glycoprotein fragment, and at least one is a MERS-CoV spike glycoprotein fragment. Any suitable SARS-CoV spike glycoprotein fragment and any suitable MERS-CoV spike glycoprotein fragment can be used. For instance, a SARS-CoV spike glycoprotein fragment can be any suitable fragment of a wild-type SARS-CoV strain, whereas a MERS-CoV spike glycoprotein fragment can be any suitable fragment of a wild-type MERS-CoV strain. Examples of such strains include SARS coronavirus Tor2 (GenBank accession number NC_004718.3) and MERS coronavirus (GenBank accession number NC_019843.3).

In some embodiments, an immunogenic fragment comprises, consists of, or consists essentially of an amino acid sequence with at least 90%, preferably at least 91%, at least 92%, at least 93%, at least 94%, at least 95%, at least 96%, at least 97%, at least 98%, or at least 99% sequence identity to a wild-type SARS-CoV or MERS spike glycoprotein, or any portion thereof.

In some embodiments, the recombinant polypeptide includes at least three immunogenic fragments, of which at least one is a SARS-CoV-2 spike glycoprotein fragment as described herein, at least one is a SARS-CoV spike glycoprotein fragment as described herein, and at least one is a MERS-CoV spike glycoprotein fragment as described herein. In some embodiments, the recombinant polypeptide includes at least three immunogenic fragments, of which at least one is a SARS-CoV-2 spike glycoprotein fragment as described herein, at least one is a SARS-CoV spike glycoprotein fragment, wherein the SARS-CoV fragment comprises, consists of, or consists essentially of an amino acid sequence with at least 90%, preferably at least 91%, at least 92%, at least 93%, at least 94%, at least 95%, at least 96%, at least 97%, at least 98%, or at least 99% sequence identity to an amino acid sequence represented by SEQ ID NO: 199 (SARS-2003, SARS_short_h), or SEQ ID NO: 201 (SARS-2003, SARS_short_i); and at least one is a MERS-CoV spike glycoprotein fragment, wherein the MERS-CoV fragment comprises, consists of, or consists essentially of an amino acid sequence with at least 90%, preferably at least 91%, at least 92%, at least 93%, at least 94%, at least 95%, at least 96%, at least 97%, at least 98%, or at least 99% sequence identity to an amino acid sequence selected from the group consisting of SEQ ID NO: 203 (MERS_Lytic_a), SEQ ID NO: 205 (MERS_Lytic_b), SEQ ID NO: 207 (MERS_Lytic_c), SEQ ID NO: 209 (MERS_Lytic_d), SEQ ID NO: 211 (MERS_Lytic_e), SEQ ID NO: 213 (MERS_Lytic_f), and SEQ ID NO: 215 (MERS_Lytic_g).

In some embodiments, the recombinant polypeptide includes at least three immunogenic fragments, of which at least one is a SARS-CoV-2 spike glycoprotein fragment as described herein, at least one is a SARS-CoV spike glycoprotein fragment, wherein the SARS-CoV fragment comprises, consists of, or consists essentially of an amino acid sequence of SEQ ID NO: 199 (SARS-2003, SARS_short_h), or SEQ ID NO: 201 (SARS-2003, SARS_short_i); and at least one is a MERS-CoV spike glycoprotein fragment, wherein the MERS-CoV fragment comprises, consists of, or consists essentially of an amino acid sequence of an amino acid sequence selected from the group consisting of SEQ ID NO: 203 (MERS_Lytic_a), SEQ ID NO: 205 (MERS_Lytic_b), SEQ ID NO: 207 (MERS_Lytic_c), SEQ ID NO: 209 (MERS_Lytic_d), SEQ ID NO: 211 (MERS_Lytic_e), SEQ ID NO: 213 (MERS_Lytic_f), and SEQ ID NO: 215 (MERS_Lytic_g).

Another embodiment is a recombinant vector that comprises, consists of, or consists essentially of, a polynucleotide that encodes the recombinant polypeptide described herein. The recombinant vector can be any suitable vector. Examples of suitable recombinant vectors include but are not limited to a pcDNA3.1, a pSV, a pCMV, a pBApo-CMV, or a pBApo-EFlalpha expression vector.

Yet another embodiment is an isolated cell that includes the recombinant polypeptide described herein or a recombinant polynucleotide that contains a nucleotide sequence that encodes the recombinant polypeptide.

Another embodiment is a pharmaceutical composition that contains the recombinant polypeptide described herein and at least one pharmaceutically acceptable carrier. The at least one pharmaceutically acceptable carrier can be any suitable carrier. Examples of suitable carriers include water and any suitable buffer. Suitable buffers include HEPES-buffered saline and phosphate-buffered saline. In certain embodiments, the pharmaceutical composition further contains at least one adjuvant. The at least one adjuvant can be any suitable adjuvant. Examples of suitable adjuvants include alum adjuvants, emulsion adjuvants, and pattern recognition receptor agonist adjuvants. Further examples include AS03, MF59, CpG, Squalene Emulsion, Alum, aluminum hydroxide gels, calcium phosphate hydroxide, paraffin oil, cytokines (IL-1, IL-2, IL-12), killed bacterial products such as Bordetella and Mycobacterium bacteria, bacterial toxoids, squalene and DL-α-tocopherol emulsions, squalene-oil-in-water emulsion, aluminum phosphate gels, saponins, cyclic dinucleotides, and TLR agonists, preferably TLR1, TLR2, TLR4, TLR5, TLR7, TLR8, TLR9 etc., and any combination thereof. In certain embodiments, the adjuvant is AS03. In certain other embodiments, the adjuvant is Alum. In yet further embodiments, the pharmaceutical composition does not contain an adjuvant.

The pharmaceutical composition can contain any therapeutically effective amount of the recombinant polypeptide described herein. A therapeutically effective amount is an amount sufficient to induce an immune response against the target virus or viruses, for instance, SARS-CoV-2, SARS-CoV, and/or MERS-CoV, preferably SARS-CoV-2. Typically, a dosage is therapeutically effective if it prevents, inhibits, reduces, eliminates, protects against, and/or delays the onset of an infection or an infectious clinical condition caused by a beta coronavirus in a subject. Infectious clinical conditions include, for example, fever or chills, cough, shortness of breath or difficult breathing, fatigue, muscle or body aches, headache, loss of taste or smell, sore throat, congestion, runny nose, nausea, vomiting, and diarrhea. In some embodiments, a single dose of the pharmaceutical composition contains 10 nanograms to 1 milligram, 0.1-250 micrograms, 10-100 micrograms, or 12.5-50 micrograms of the recombinant polypeptide. In some embodiments, a single dose of the pharmaceutical composition contains 0.01-1, 0.1-1, 0.5-5, 1-20, or 5-15, 1-50, or 10-50 micrograms of the recombinant polypeptide. In some embodiments, wherein the pharmaceutical composition does not contain an adjuvant, a single dose of the pharmaceutical composition can contain 0.01-1, 0.1-1, 0.5-5, 1-20, or 5-15, 1-50, or 10-50 micrograms of the recombinant polypeptide. However, a single dose of the pharmaceutical composition can also contain increased amounts of the recombinant polypeptide, such as 100-1000, 100-250, or 250-500 micrograms of recombinant polypeptide.

A further embodiment is a method for preventing, inhibiting, reducing, eliminating, protecting against, or delaying the onset of an infection or an infectious clinical condition caused by a beta coronavirus in a subject comprising administering to the subject the recombinant polypeptide described herein, the polypeptide encoded by the recombinant polynucleotide described herein, or a dose of the pharmaceutical composition described herein. Examples of beta coronaviruses include SARS-CoV, MERS-CoV, and SARS-CoV-2.

Another embodiment is a method for inducing an immune response against a beta coronavirus in a subject comprising administering to the subject the recombinant polypeptide described herein, the polypeptide encoded by the recombinant polynucleotide described herein, or a dose of the pharmaceutical composition described herein. Examples of beta coronaviruses include SARS-CoV, MERS-CoV, and SARS-CoV-2.

In the methods described herein, administration can be achieved by any suitable administration method. Suitable administration methods include oral, parenteral, subcutaneous, intradermal, transdermal, intravenous, intramuscular, intrapulmonary, intranasal, intraarterial, intrathecal, and intraperitoneal administration.

In the methods described herein, the subject can be any suitable animal that is capable of being infected by a beta coronavirus, such as SARS-CoV, MERS-CoV, and SARS-CoV-2. The subject can be a human, non-human primate, horse, pig, cattle, cat, dog, sheep, mink, rodent, hamster, or bat, preferably human.

### Examples of Non-Limiting Aspects of the Disclosure

Aspects, including embodiments, of the invention described herein may be beneficial alone or in combination, with one or more other aspects or embodiments. Without limiting the foregoing description, certain non-limiting aspects of the disclosure numbered (1)-(41) are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A recombinant polypeptide comprising at least one immunogenic fragment of Severe Acute Respiratory Syndrome Coronavirus 2 (SARS-CoV-2) spike glycoprotein, wherein each immunogenic fragment comprises an N-terminus region, wherein the N-terminus region comprises an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355.

(2) The recombinant polypeptide of aspect 1, wherein each immunogenic fragment further comprises a C-terminus region, and the C-terminus region comprises an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423.

(3) A recombinant polypeptide comprising at least one immunogenic fragment of SARS-CoV-2 spike glycoprotein, wherein each immunogenic fragment comprises an N-terminus region and a C-terminus region, and wherein the N-terminus region comprises an amino acid sequence that differs at at least 2 residues from an amino acid sequence selected from the group consisting of SEQ ID NOs: 245-289 and 293-362, and amino acid sequences DYS, YS, and S, preferably selected from SEQ ID NOs: 245-248, 250-251, 255-261, 256-258, 293-296, 298-299, 303-309, 304-306, 314-317, 319-320, 324-330, 325-327, 342-345, 347-348, 352-358, and 353-355, and the C-terminus region comprises an amino acid sequence that differs at at least 2 residues from an amino acid sequence selected from the group consisting of SEQ ID NOs: 363-398 and 402-423, and amino acid sequences STN, ST, and S, preferably selected from SEQ ID NOs: 382-397, 382-387, 388-393, 394-397, 385-390, 391-395, and 421-423.

(4) A recombinant polypeptide comprising at least one immunogenic fragment of SARS-CoV-2 spike glycoprotein, wherein each immunogenic fragment comprises an N-terminus region and a C-terminus region, and wherein the N-terminus region comprises an amino acid sequence of at least five contiguous amino acid residues of an amino acid sequence selected from the group consisting of SEQ ID NOs: 245, 293, 314, and 342, and the C-terminus region comprises an amino acid sequence of at least five contiguous amino acid residues of an amino acid sequence of SEQ ID NO: 363 or 402.

(5) The recombinant polypeptide of aspect 4, wherein each immunogenic fragment comprises an N-terminus region and a C-terminus region, and wherein the N-terminus region comprises an amino acid sequence of at least ten contiguous amino acid residues of an amino acid sequence selected from the group consisting of SEQ ID NOs: 245, 293, 314, and 342 and the C-terminus region comprises an amino acid sequence of at least ten contiguous amino acid residues of an amino acid sequence of SEQ ID NO: 363 or 402.

(6) The recombinant polypeptide of any one of aspects 1-5, wherein each immunogenic fragment comprises an intervening region between the N-terminus region and the C-terminus region, and wherein each intervening region comprises an amino acid sequence selected from the group consisting of SEQ ID NOs: 2045-2050 or that differs at at least 4 residues from an amino acid sequence selected from the group consisting of SEQ ID NOs: 603-606, and 2037-2040.

(7) The recombinant polypeptide of any one of aspects 1-6, wherein each of the at least one immunogenic fragments comprises an amino acid sequence with at least 95% sequence identity to an amino acid sequence independently selected from the group consisting of SEQ ID NOs: 1577-1661 and 1917-1946.

(8) The recombinant polypeptide of any of one aspects 1-7, wherein the polypeptide comprises at least two immunogenic fragments.

(9) The recombinant polypeptide of any one of aspects 1-8, wherein each immunogenic fragment of the at least one immunogenic fragments comprises the same amino acid sequence.

(10) The recombinant polypeptide of any one of aspects 1-8, wherein each immunogenic fragment of the at least one immunogenic fragment comprises a different amino acid sequence from each of the other immunogenic fragments.

(11) The recombinant polypeptide of any one of aspects 1-10, wherein the recombinant polypeptide is expressed at a concentration of at least 45 mg protein/L culture.

(12) The recombinant polypeptide of any one of aspects 1-11, wherein the recombinant polypeptide exhibits at least 85% protease resistance.

(13) The recombinant polypeptide of any one of aspect 1-14, wherein the recombinant polypeptide does not comprise an amino acid sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 217, 219, 221, 223, 225, 227, 229, 231, 233, 235, 237, 239, 241, 243, and 2057-2066.

(14) The recombinant polypeptide of any one of aspects 1-13, wherein the recombinant polypeptide further comprises an antibody Fc region.

(15) The recombinant polypeptide of any one of aspects 1-14, wherein the polypeptide comprises at least two immunogenic fragments, and wherein the at least two immunogenic fragments are connected to each other via a linker.

(16) The recombinant polypeptide of aspect 15, wherein the linker is a polypeptide comprising an amino acid sequence of 1-35 residues, wherein each residue is independently serine or glycine.

(17) The recombinant polypeptide of any one of aspects 14-16, wherein the at least one immunogenic fragment of the SARS-CoV-2 spike glycoprotein is connected to the antibody Fc region via a linker.

(18) The recombinant polypeptide of aspect 17, wherein the linker comprises an amino acid sequence selected from the group consisting of SEQ ID NOs: 65 (Fc1), SEQ ID NO: 67 (Fc1-TEV), SEQ ID NO: 69 (Fc1-Rv3C), SEQ ID NO: 193 (Short), SEQ ID NO: 195 (Medium), and SEQ ID NO: 197 (Long).

(19) The recombinant polypeptide of any one of aspects 14-18, wherein the antibody Fc region is from a human IgG1 antibody or derived therefrom.

(20) The recombinant polypeptide of aspect 19, wherein the antibody Fc region comprises the amino acid sequence of SEQ ID NO: 71.

(21) The recombinant polypeptide of any one of aspects 1-20, wherein the polypeptide comprises an amino acid sequence with at least 95% sequence identity to an amino acid sequence selected from the group consisting of SEQ ID NOs: 1662-1746 and 1947-1976.

(22) The recombinant polypeptide of aspect 21, wherein the polypeptide comprises an amino acid sequence selected from the group consisting of SEQ ID NOs: 1662-1746 and 1947-1976.

(23) A recombinant polynucleotide encoding the polypeptide of any one of aspects 1-22.

(24) The recombinant polynucleotide of aspect 23, wherein the polynucleotide comprises a nucleic acid sequence with at least 90%, preferably at least 91%, 92%, 93%, 94%, 95%, 96%, 97%, 98%, or 99%, sequence identity to a nucleic acid sequence selected from the group consisting of SEQ ID NOs: 1832-1916 and 2007-2036.

(25) The recombinant polynucleotide of aspect 24, wherein the polynucleotide comprises a nucleic acid sequence selected from the group consisting of SEQ ID NOs: 1832-1916 and 2007-2036.

(26) The recombinant polynucleotide of any one of aspects 23-25, wherein the nucleic acid sequence has been codon optimized.

(27) A pharmaceutical composition comprising the recombinant polypeptide of any one of aspects 1-22 or the polypeptide encoded by the recombinant polynucleotide of any one of aspects 23-26, and a pharmaceutically acceptable carrier.

(28) The pharmaceutical composition of aspect 27, wherein the pharmaceutical composition further comprises at least one adjuvant.

(29) The pharmaceutical composition of aspect 27, wherein the pharmaceutical composition does not comprise an adjuvant.

(30) The pharmaceutical composition of aspect 28, wherein the at least one adjuvant is selected from the group consisting of alum adjuvants, emulsion adjuvants, and pattern recognition receptor agonist adjuvants.

(31) The pharmaceutical composition of aspect 28, wherein the at least one adjuvant is AS03, MF59, CpG, Squalene Emulsion, Alum, aluminum hydroxide gels, calcium phosphate hydroxide, paraffin oil, cytokines (IL-1, IL-2, IL-12), killed bacterial products such as Bordetella and Mycobacterium bacteria, bacterial toxoids, squalene and DL-α-tocopherol emulsions, aluminum phosphate gels, saponins, cyclic dinucleotides, and TLR agonists, preferably TLR1, TLR2, TLR4, TLR5, TLR7, TLR8, TLR9 etc., and any combination thereof.

(32) The pharmaceutical composition of aspect 28, wherein the at least one adjuvant is a squalene-oil-in-water emulsion adjuvant.

(33) A vector comprising the recombinant polynucleotide of any one of aspects 23-26.

(34) An isolated cell comprising the recombinant polypeptide of any one of aspects 1-22, or the polypeptide encoded by the recombinant polynucleotide of any one of aspects 23-26.

(35) A method for preventing, inhibiting, reducing, eliminating, protecting, or delaying the onset of an infection or an infectious clinical condition caused by a beta coronavirus in a subject comprising administering to the subject the recombinant polypeptide of any one of aspects 1-22, the polypeptide encoded by the recombinant polynucleotide of any one of aspects 23-26, or the pharmaceutical composition of any one of aspects 27-32.

(36) A method for inducing an immune response against a beta coronavirus in a subject comprising administering to the subject the recombinant polypeptide of any one of aspects 1-22, the polypeptide encoded by the recombinant polynucleotide of any one of aspects 23-26, or the pharmaceutical composition of any one of aspects 27-32.

(37) The method of aspect 35 or 36, wherein the recombinant polypeptide of any one of aspects 1-22, the polypeptide encoded by the recombinant polynucleotide of any one of aspects 23-26, or the pharmaceutical composition of any one of aspects 27-32 is administered by oral, parenteral, subcutaneous, intradermal, transdermal, intravenous, intramuscular, intranasal, intrapulmonary, intraarterial, intrathecal, or interperitoneal administration.

(38) The method of any one of aspects 35-37, wherein the coronavirus is selected from the group consisting of SARS-CoV-2, SARS-CoV, and MERS-CoV

(39) The method of any one of aspects 35-38, wherein the subject is a mammal.

(40) The method of any one of aspects 35-39, wherein the mammal is a human or non-human primate.

(41) The use of the recombinant polypeptide of any one of aspects 1-22, the polypeptide encoded by the recombinant polynucleotide of any one of aspects 23-26, or the pharmaceutical composition of any one of aspects 27-32 for the preparation of a medicament for the treatment or prevention of illness caused by SARS-CoV-2.

(42) The recombinant polypeptide of any one of aspects 1-22, the polypeptide encoded by the recombinant polynucleotide of any one of aspects 23-26, or the pharmaceutical composition of any one of aspects 27-32 for use as a medicament.

### EXAMPLES

### Example 1

Cloning: Inserts encoding a certain fragment of SARS CoV-2 virus Spike protein S1 were designed. The inserts were codon optimized for CHO cell expression using IDTDNA codon optimization algorithms. The insert was cloned using either (a) restriction digests and DNA ligations or (b) NEB HiFi DNA assembly builder mix into a pcDNA3.1 vector containing appropriate secretion signal sequences, linkers, and tags for secreted fusion protein expression, as well as sequence encoding a human IgG1 Fc region. The resulting clone encoding a recombinant SARS CoV-2 Spike protein fragment—Fc region fusion protein (“recombinant CoV-2 fusion protein”) was verified by either colony PCR and/or restriction digests. DNA sequencing was also used.

Protein Expression: Supercoiled plasmid of the verified clone was transiently transfected into CHO-S cells and expressed under the control of a constitutive promoter within cell culture conditions ranging from 30-37° C. and 3-10 days in CO₂ (8%) incubators with rotary shaking agitation at speeds of 150 RPM. Cells were removed by centrifugation and culture medium containing the recombinant CoV-2 fusion protein were passed over Protein A agarose to bind the Fc region-containing CoV-2 protein. Filtration could also have been used to remove cells. The column containing bound recombinant CoV-2 fusion protein was washed with phosphate buffered saline. The recombinant CoV-2 fusion protein bound to the Protein A column was eluted with low pH glycine followed by neutralization in pH8.0 Tris. The recombinant CoV-2 fusion protein was dialyzed to remove glycine/tris and placed into HEPES-buffered saline (10 mM HEPES. 150 mM NaCl, pH adjusted with NaOH to pH 7.2-7.5). No additional purification was employed in this case. However, additional purification by any chromatography method such as HIC or ion exchange can optionally be used.

Following protein expression in accordance with the description above, resulting protein was analyzed by reducing SDS-PAGE to determine protein integrity for monomeric CTD-Fcs. Protein yields were calculated using absorbance of 280 nm light and protein-specific extinction coefficients determined in silico according to their expected amino acid composition. Representative protein yields are given in Table 3.

TABLE 3

| | Immunogenic | Expression (mg protein/ | |
|---|---|---|---|
| Strain(s) | Fragment | L CHO culture (±SD)) | |
| 3472 (SEQ ID NO: 75) | CTD_long_a | 77.3 | ⁽⁵⁾ |
| | (SEQ ID NO: 1) | | |
| 2356 (SEQ ID NO: 101) | CTD_long_b | 60.8 | ⁽⁵⁾ |
| | (SEQ ID NO: 7) | | |
| 2357 (SEQ ID NO: 103) | CTD_long_c | 60.8 | ⁽⁵⁾ |
| | (SEQ ID NO: 9) | | |
| 2358 (SEQ ID NO: 105) | CTD_long_d | 65.8 | ⁽⁵⁾ |
| | (SEQ ID NO: 11) | | |
| 2359 (SEQ ID NO: 107) | CTD_long_e | 96.1 | ⁽⁵⁾ |
| | (SEQ ID NO: 13) | | |
| 2360 (SEQ ID NO: 109) | CTD_long_f | 95.1 | ⁽⁵⁾ |
| | (SEQ ID NO: 15) | | |
| 2361 (SEQ ID NO: 111) | CTD_long_g | 74.5 | ⁽⁵⁾ |
| | (SEQ ID NO: 17) | | |
| 2362 (SEQ ID NO: 113) | CTD_long_h | 108.3 | ⁽⁵⁾ |
| | (SEQ ID NO: 19) | | |
| 2363 (SEQ ID NO: 115) | CTD_long_i | 108.5 | ⁽⁵⁾ |
| | (SEQ ID NO: 21) | | |
| 2330 (SEQ ID NO: 73) | CTD_short_a | >40 | ⁽⁵⁾ |
| | (SEQ ID NO: 23) | | |
| 2364 (SEQ ID NO: 117) | CTD_short_b | 131.8 | ⁽⁵⁾ |
| | (SEQ ID NO: 25) | | |
| 2365 (SEQ ID NO: 119) | CTD_short_c | 126.5 | ⁽⁵⁾ |
| | (SEQ ID NO: 27) | | |
| 2366 (SEQ ID NO: 121) | CTD_short_d | 106.2 | ⁽⁵⁾ |
| | (SEQ ID NO: 29) | | |
| 2367 (SEQ ID NO: 123) | CTD_short_e | 107.5 | ⁽⁵⁾ |
| | (SEQ ID NO: 31) | | |
| 2368 (SEQ ID NO: 125) | CTD_short_f | 109.9 | ⁽⁵⁾ |
| | (SEQ ID NO: 33) | | |
| 2369 (SEQ ID NO: 127) | CTD_short_g | 132.4 | ⁽⁵⁾ |
| | (SEQ ID NO: 35) | | |
| 2370 (SEQ ID NO: 129) | CTD_short_h | 67.5 | ⁽⁵⁾ |
| | (SEQ ID NO: 37) | | |
| 2371 (SEQ ID NO: 131) | CTD_short_i | 167.7 | ⁽⁵⁾ |
| | (SEQ ID NO: 39) | | |
| 2372 (SEQ ID NO: 133) | CTD_vs_a | 2.3 | ⁽⁵⁾ |
| | (SEQ ID NO: 41) | | |
| 2373 (SEQ ID NO: 135) | CTD_vs_b | 1.7 | ⁽⁵⁾ |
| | (SEQ ID NO: 43) | | |
| 2374 (SEQ ID NO: 137) | CTD_vs_c | 0.9 | ⁽⁵⁾ |
| | (SEQ ID NO: 45) | | |
| 2375 (SEQ ID NO: 139) | CTD_vs_d | ≤0.8 | ⁽⁵⁾ |
| | (SEQ ID NO: 47) | | ⁽⁵⁾ |
| 2376 (SEQ ID NO: 141) | CTD_vs_e | ≤0.8 | |
| | (SEQ ID NO: 49) | | |
| 2377 (SEQ ID NO: 143) | RBD_a | ≤0.8 | ⁽⁵⁾ |
| | (SEQ ID NO: 51) | | |
| 2378 (SEQ ID NO: 145) | RBD_b | ≤0.8 | ⁽⁵⁾ |
| | (SEQ ID NO: 53) | | |
| 2380 (SEQ ID NO: 149) | RBD_d | ≤0.8 | ⁽⁵⁾ |
| | (SEQ ID NO: 55) | | |
| 2381 (SEQ ID NO: 151) | RBD_e | 14.5 | ⁽⁵⁾ |
| | (SEQ ID NO: 57) | | |

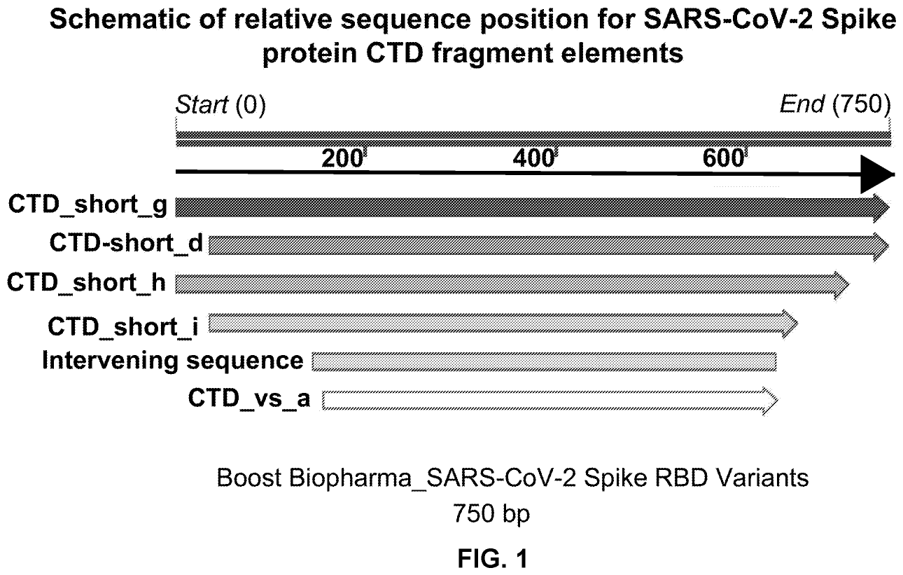

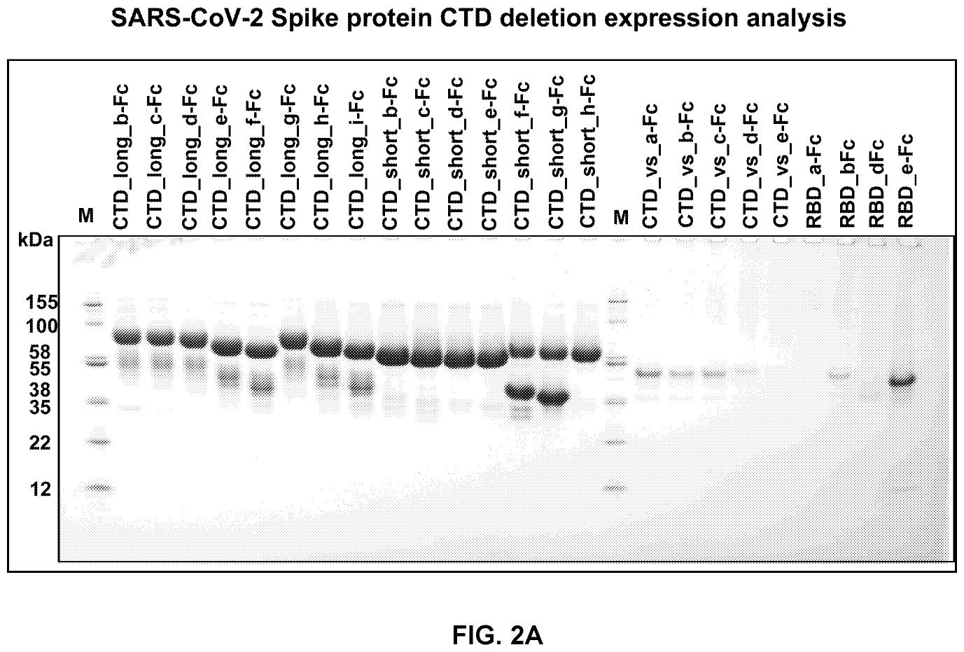

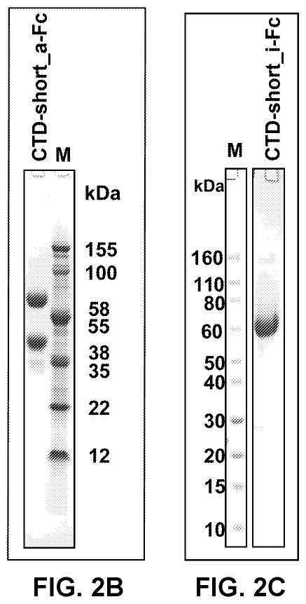

As can be seen in Table 3 and FIGS. 1A-B, protein expression varies significantly depending on what immunogenic fragment the strain encodes. It was surprisingly found that the amino acid sequence of the endpoints of certain immunogenic fragments was associated with high protein expression, in some cases more than 100-fold higher expression than immunogenic fragments containing different amino acid endpoint sequences.

### Example 2

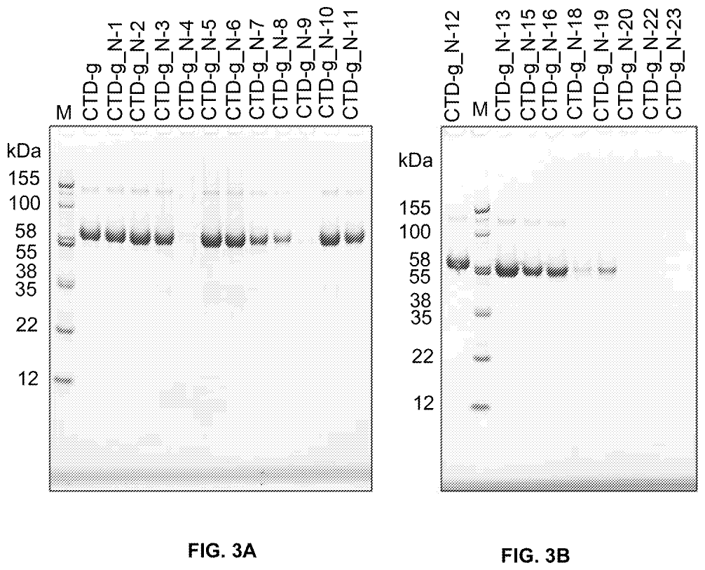

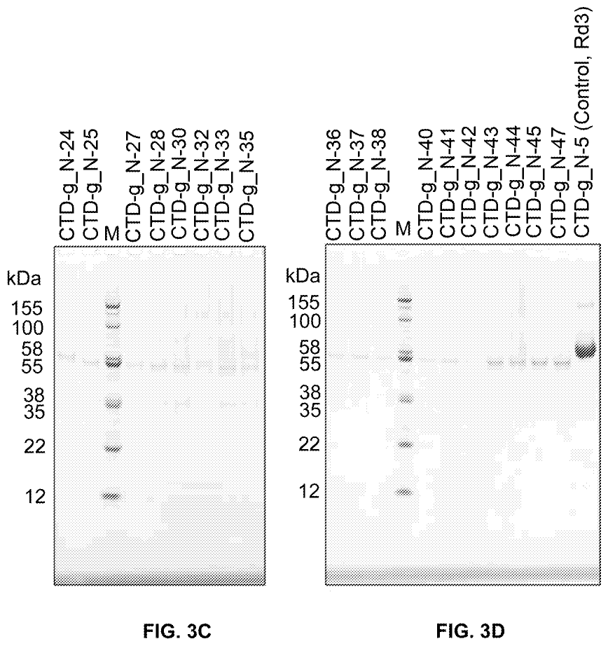

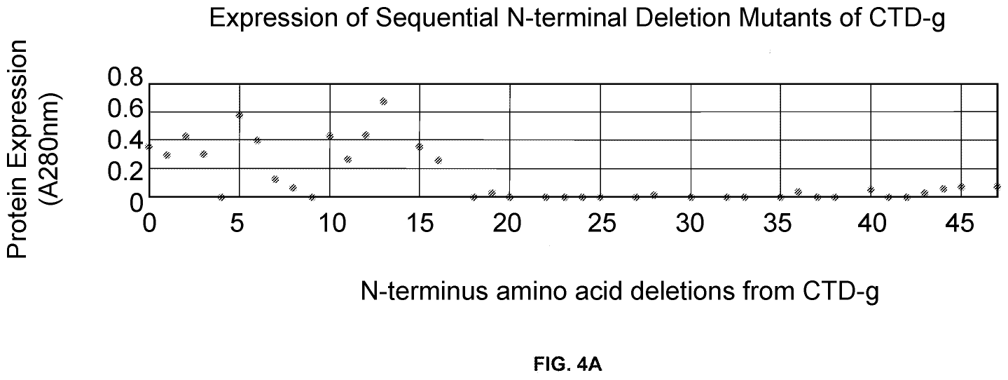

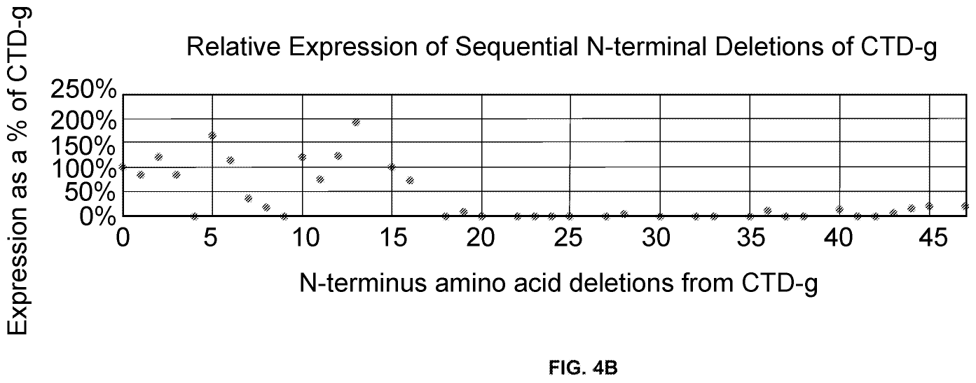

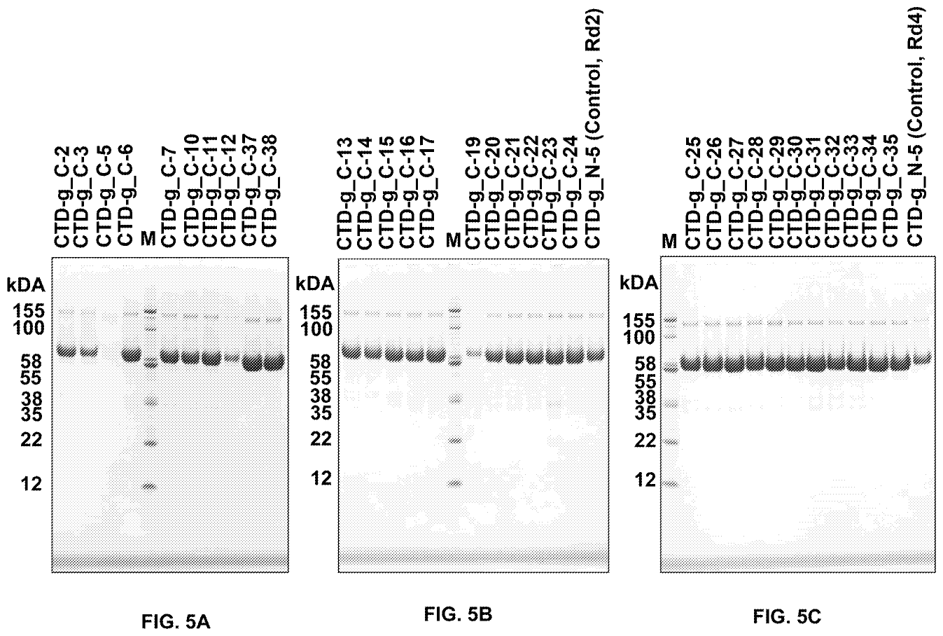

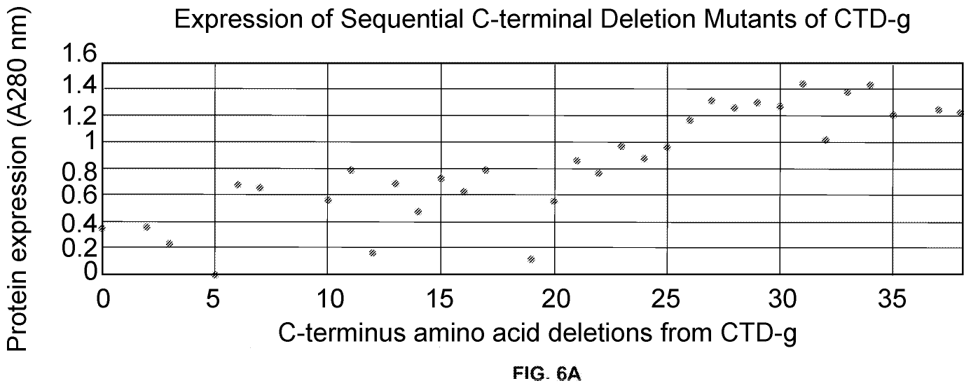

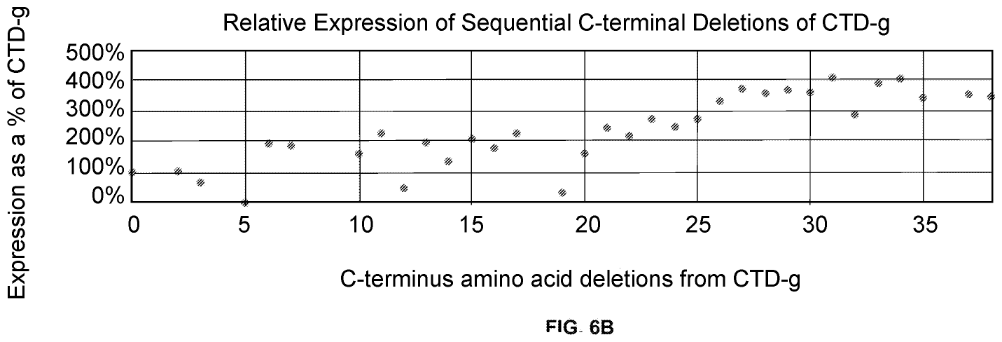

Expression construct CDS inserts were made by DNA synthesis and cloned into a C-terminal linker-Fc (human IgG1 Fc) expression vector. Transfection grade plasmid was prepared and used for transient transfection of ExpiCHO-S cells using the Mirus Bio CHOgro medium and transfection system. Transfection cultures were on the 25 mL scale. After 7-days of post-transfection culturing, protein was affinity purified from the cell medium using Protein A chromatography, washed extensively with PBS (pH 7.2), and eluted with low-pH sodium acetate elution followed by rapid neutralization with TRIS/HCl buffer, pH7.2. Eluted protein was analyzed by spectrophotometric absorbance of 280 nm light (data in table 4, graphed in FIGS. 4A, 41B, 6A, and 6B) and by reducing SDS-PAGE (shown in FIGS. 3A-D and 5A-C). All transfections, purifications, and SDS-PAGE sample load volumes were done with the same volumes and thus allow direct comparison of between samples. The starting protein is CTD-short_g, the sequence of which is [SEQ ID NO: 245]-[SEQ ID NO: 603]-[SEQ ID NO: 363]. For each N-terminal deletion listed in Table 4, i.e., CTD-g_N-1—CTD-g_N-47, the sequence of each tested immunogenic fragment is [the sequence corresponding to CTD-g_N-[#] as shown in Table 4]-[SEQ ID NO: 603]-[SEQ TD NO: 363]. For each C-terminal deletion, i.e., CTD-g_C-2—CTD-g_C-38, the sequence ofeach tested immunogenic fragment is [SEQ TD NO: 245]-[SEQ ID NO: 603]-[the sequence corresponding to CTD-g_C-[#] as shown in Table 4].

TABLE 4

| | Protein Name | | | Protein Name | |
|---|---|---|---|---|---|
| | (SEQ ID NO of | | | (SEQ ID NO of | |
| | corresponding N- | A280 | | corresponding C- | A280 |
| Note | terminus region) | (Nanodrop) | Note | terminus region) | (Nanodrop) |
| Start | CTD-g (SEQ ID | 0.35 | C-terminal | CTD-g_C-2 (SEQ | 0.359 |
| Protein | NO: 245) | | deletions | ID NO 365) | |
| (CTD- | | | | | |
| short_g); | | | | | |
| RBD N- | | | | | |
| terminus | | | | | |
| for Ren, et | | | | | |
| al., Nature, | | | | | |
| 2020 | | | | | |
| N-terminal | CTD-g_N-1 (SEQ | 0.296 | | CTD-g_C-3 (SEQ | 0.233 |
| deletions | ID NO: 246) | | | ID NO: 366) | |
| | CTD-g_N-2 (SEQ | 0.425 | | CTD-g_C-5 (SEQ | 0 |
| | ID NO: 247) | | | ID NO: 368) | |
| | CTD-g_N-3 (SEQ | 0.298 | | CTD-g_C-6 (SEQ | 0.683 |
| | ID NO: 248) | | | ID NO: 369) | |
| | CTD-g_N-4 (SEQ | 0 | | CTD-g_C-7 (SEQ | 0.659 |
| | ID NO: 249) | | | ID NO: 370) | |
| | CTD-g_N-5 (SEQ | 0.574 | | CTD-g_C-10 | 0.564 |
| | ID NO: 250) | | | (SEQ ID NO: 373) | |
| | CTD-g_N-6 (SEQ | 0.4 | | CTD-g_C-11 | 0.792 |
| | ID NO: 251) | | | (SEQ ID NO: 374) | |
| | CTD-g_N-7 (SEQ | 0.126 | | CTD-g_C-12 | 0.168 |
| | ID NO: 252) | | | (SEQ ID NO: 375) | |
| | CTD-g_N-8 (SEQ | 0.065 | | CTD-g_C-13 | 0.69 |
| | ID NO: 253) | | | (SEQ ID NO: 376) | |
| | CTD-g_N-9 (SEQ | 0 | | CTD-g_C-14 | 0.48 |
| | ID NO: 254) | | | (SEQ ID NO: 377) | |
| | CTD-g_N-10 | 0.428 | | CTD-g_C-15 | 0.725 |
| | (SEQ ID NO: 255) | | | (SEQ ID NO: 378) | |
| | CTD-g_N-11 | 0.261 | | CTD-g_C-16 | 0.627 |
| | (SEQ ID NO: 256) | | | (SEQ ID NO: 379) | |
| N-terminus | CTD-g_N-12 | 0.434 | | CTD-g_C-17 | 0.792 |
| of CTD- | (SEQ ID NO: 257) | | | (SEQ ID NO: 380) | |
| short_i | | | | | |
| | CTD-g_N-13 | 0.67 | | CTD-g_C-19 | 0.114 |
| | (SEQ ID NO: 258) | | | (SEQ ID NO: 382) | |
| | CTD-g_N-15 | 0.354 | | CTD-g_C-20 | 0.558 |
| | (SEQ ID NO: 260) | | | (SEQ ID NO: 383) | |
| | CTD-g_N-16 | 0.253 | | CTD-g_C-21 | 0.861 |
| | (SEQ ID NO: 261) | | | (SEQ ID NO: 384) | |
| | CTD-g_N-18 | 0 | | CTD-g_C-22 | 0.764 |
| | (SEQ ID NO: 263) | | | (SEQ ID NO: 385) | |
| | CTD-g_N-19 | 0.03 | | CTD-g_C-23 | 0.968 |
| | (SEQ ID NO: 264) | | | (SEQ ID NO: 386) | |
| | CTD-g_N-20 | 0 | | CTD-g_C-24 | 0.872 |
| | (SEQ ID NO: 265) | | | (SEQ ID NO: 387) | |
| | CTD-g_N-22 | 0 | | CTD-g_C-25 | 0.964 |
| | (SEQ ID NO: 267) | | | (SEQ ID NO: 388) | |
| | CTD-g_N-23 | 0 | | CTD-g_C-26 | 1.164 |
| | (SEQ ID NO: 268) | | | (SEQ ID NO: 389) | |
| | CTD-g_N-24 | 0 | C-terrminus of | CTD-g_C-27 | 1.315 |
| | (SEQ ID NO: 269) | | RBD (Ren, et | (SEQ ID NO: 390) | |
| | | | al., Nature, | | |
| | | | 2020) | | |
| | CTD-g_N-25 | 0 | | CTD-g_C-28 | 1.255 |
| | (SEQ ID NO: 270) | | | (SEQ ID NO: 391) | |
| | CTD-g_N-27 | 0 | | CTD-g_C-29 | 1.295 |
| | (SEQ ID NO: 272) | | | (SEQ ID NO: 392) | |
| | CTD-g_N-28 | 0.013 | | CTD-g_C-30 | 1.268 |
| | (SEQ ID NO: 273) | | | (SEQ ID NO: 393) | |
| | CTD-g_N-30 | 0 | C-terminus of | CTD-g_C-31 | 1.44 |
| | (SEQ ID NO: 275) | | CTD-short_i | (SEQ ID NO: 394) | |
| | CTD-g_N-32 | 0 | | CTD-g_C-32 | 1.015 |
| | (SEQ ID NO: 277) | | | (SEQ ID NO: 395) | |
| | CTD-g_N-33 | 0 | | CTD-g_C-33 | 1.38 |
| | (SEQ ID NO: 278) | | | (SEQ ID NO: 396) | |
| | CTD-g_N-35 | 0 | | CTD-g_C-34 | 1.427 |
| | (SEQ ID NO: 280) | | | (SEQ ID NO: 397) | |
| | CTD-g_N-36 | 0.037 | | CTD-g_C-35 | 1.206 |
| | (SEQ ID NO: 281) | | | (SEQ ID NO: 398) | |
| | CTD-g_N-37 | 0 | | CTD-g_C-37 | 1.241 |
| | (SEQ ID NO: 282) | | | (SEQ ID NO: 400) | |
| | CTD-g_N-38 | 0 | | CTD-g_C-38 | 1.217 |
| | (SEQ ID NO: 283) | | | (SEQ ID NO: 401) | |
| | CTD-g_N-40 | 0.046 | | | |
| | (SEQ ID NO: 285) | | | | |
| | CTD-g_N-41 | 0 | Transfection Controls | | |
| | (SEQ ID NO: 286) | | | | |
| | CTD-g_N-42 | 0 | Round 2 | CTD-g_N-5 (SEQ | 0.664 |
| | (SEQ ID NO: 287) | | | ID NO: 250) | |
| | CTD-g_N-43 | 0.023 | Round 3 | CTD-g_N-5 (SEQ | 0.477 |
| | (SEQ ID NO: 288) | | | ID NO: 250) | |
| | CTD-g_N-44 | 0.057 | Round 4 | CTD-g_N-5 (SEQ | 0.392 |
| | (SEQ ID NO: 289) | | | ID NO: 250) | |
| | CTD-g_N-45 | 0.073 | | | |
| | (SEQ ID NO: 290) | | | | |
| | CTD-g_N-47 | 0.073 | | | |
| | (SEQ ID NO: 292) | | | | |

### Example 3

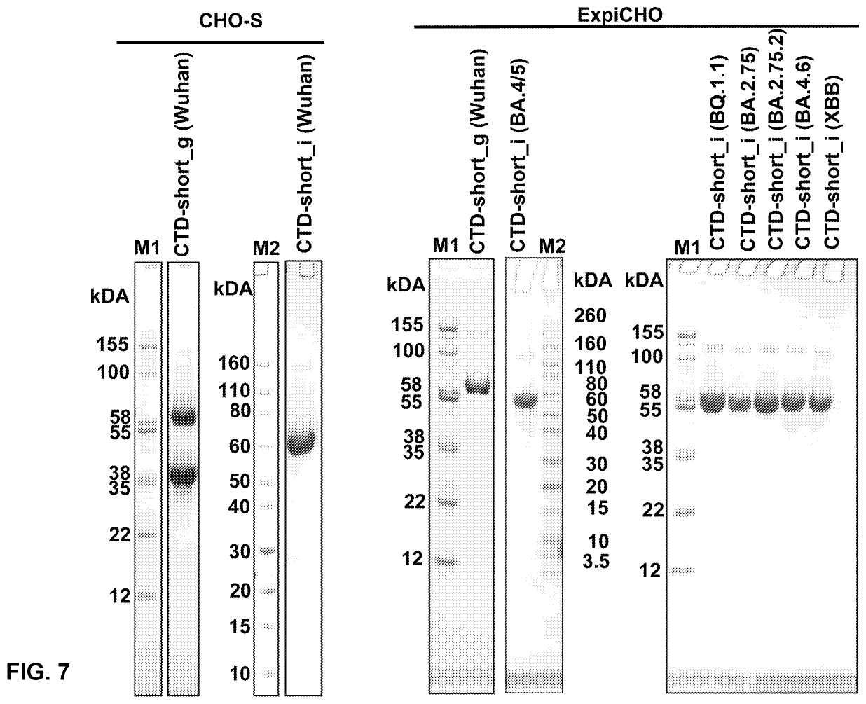

The expression constructs shown in FIG. 7 were prepared and analyzed in accordance with the protocol used in Example 2, with the exception that the transfected cells were either CHO-S or ExpiCHO cells, as shown in the figure. Particularly, the constructs analyzed were CTD-short_g (Wuhan) (the sequence of which is [SEQ ID NO: 245]-[SEQ ID NO: 603]-[SEQ ID NO: 363]), and CTD-short_i (Wuhan) (the sequence of which is [SEQ ID NO: 257]—[SEQ ID NO: 603]-[SEQ ID NO: 394]), both of which were grown in CHO-S cells. CTD-short_i (BA.4/5) (the sequence of which is [SEQ ID NO: 257]—[SEQ ID NO: 2047]-[SEQ ID NO: 394]); CTD-short_i (BQ.1.1) (the sequence of which is [SEQ ID NO: 257]—[SEQ ID NO: 2045]-[SEQ ID NO: 394]); CTD-short_i (BA.2.75) (the sequence of which is [SEQ ID NO: 257]-[SEQ ID NO: 2048]-[SEQ ID NO: 394]); CTD-short_i (BA.2.75.2) (the sequence of which is [SEQ ID NO: 257]—[SEQ ID NO: 2049]-[SEQ ID NO: 394]); CTD-short_i (BA.4.6) (the sequence of which is [SEQ ID NO: 257]—[SEQ ID NO: 2050]-[SEQ ID NO: 394]); and CTD-short_i (XBB) (the sequence of which is [SEQ ID NO: 257]—[SEQ ID NO: 2046]-[SEQ ID NO: 394]), were all grown in ExpiCHO cells. As can be seen in FIG. 7, CTD-short_i ends allow for stable expression of the resulting fragment. SARS-CoV-2 Spike RBD-containing regions with ends defined by CTD-short g and CTD-short_i exhibit differential expressibility and stability, insofar as CTD-short_i constructs demonstrate superior stability to CTD-short_g constructs independent of which variant, e.g., BA.4/5, BQ.1.1, and XBB, the CTD-short_i construct contains.

### Example 4

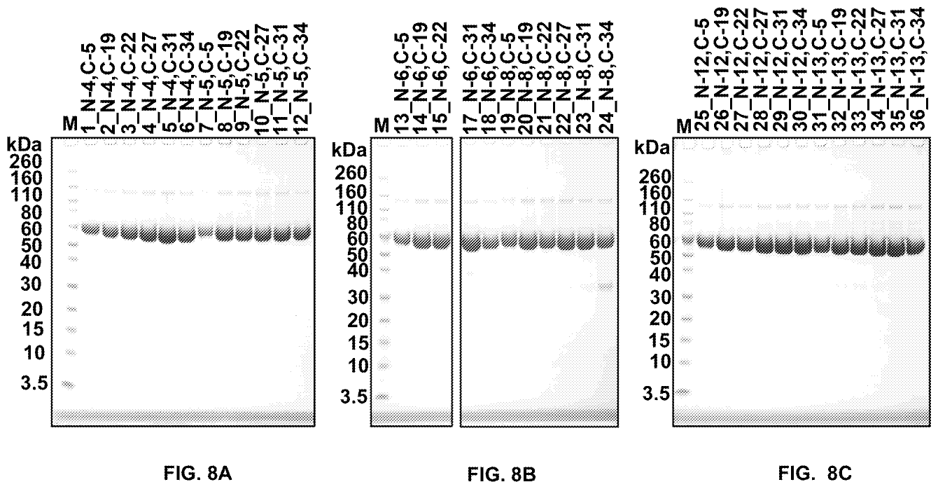

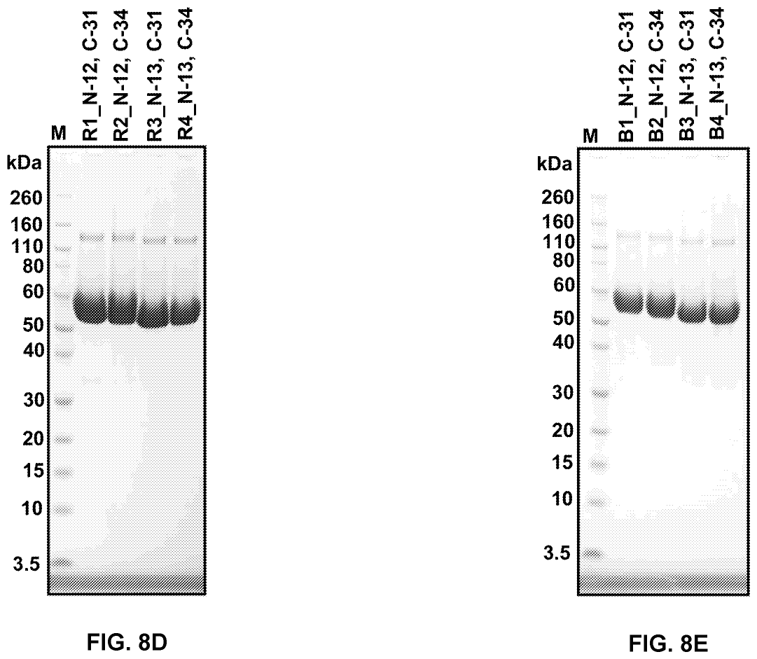

The expression constructs shown in FIGS. 8A-E and Table 5 below were prepared and analyzed in accordance with the protocol used in Example 2, with the exception that the constructs were cultured in CHO cells for 6 days rather than 7. The sequence of each construct is as follows: [“N-term” sequence]-[“Intervening Sequence” ]-[“C-term” sequence]. For example, the sequence of Construct 1 is the combined sequence of SEQ ID NO: 249-SEQ ID NO: 603-SEQ ID NO: 368.

As can be seen in Table 5, superior expression is seen in constructs with an N-terminus between N-4 and N-13 and a C-terminus between C-19 and C-34. When the endpoints are both within these ranges, then expression is surprisingly and substantially increased in comparison to when either endpoint is outside of these ranges, e.g., when the C-terminus is C-5.

TABLE 5

| | | Intervening | C-term | |
|---|---|---|---|---|
| | | Sequence | (SEQ | |
| Construct | N-term | (SEQ ID NO | ID NO | |
| No. in | (SEQ ID NO | for AA | for AA | |
| Figure | for AA seq.) | seq.) | seq.) | A280 nm |
| 1 | N-4 (249) | 603 | C-5 (368) | 0.478 |
| 2 | N-4 (249) | 603 | C-19 (382) | 0.615 |
| 3 | N-4 (249) | 603 | C-22 (385) | 0.858 |
| 4 | N-4 (249) | 603 | C-27 (390) | 1.061 |
| 5 | N-4 (249) | 603 | C-31 (394) | 1.438 |
| 6 | N-4 (249) | 603 | C-34 (397) | 0.988 |
| 7 | N-5 (250) | 603 | C-5 (368) | 0.313 |
| 8 | N-5 (250) | 603 | C-19 (382) | 1.038 |
| 9 | N-5 (250) | 603 | C-22 (385) | 0.810 |
| 10 | N-5 (250) | 603 | C-27 (390) | 0.894 |
| 11 | N-5 (250) | 603 | C-31 (394) | 0.948 |
| 12 | N-5 (250) | 603 | C-34 (397) | 0.983 |
| 13 | N-6 (251) | 603 | C-5 (368) | 0.485 |
| 14 | N-6 (251) | 603 | C-19 (382) | 1.278 |
| 15 | N-6 (251) | 603 | C-22 (385) | 1.168 |
| 17 | N-6 (251) | 603 | C-31 (394) | 1.589 |
| 18 | N-6 (251) | 603 | C-34 (397) | 0.751 |
| 19 | N-8 (253) | 603 | C-5 (368) | 0.668 |
| 20 | N-8 (253) | 603 | C-19 (382) | 1.338 |
| 21 | N-8 (253) | 603 | C-22 (385) | 0.953 |
| 22 | N-8 (253) | 603 | C-27 (390) | 1.408 |
| 23 | N-8 (253) | 603 | C-31 (394) | 1.239 |
| 24 | N-8 (253) | 603 | C-34 (397) | 0.911 |
| 25 | N-12 (257) | 603 | C-5 (368) | 0.681 |
| 26 | N-12 (257) | 603 | C-19 (382) | 1.176 |
| 27 | N-12 (257) | 603 | C-22 (385) | 1.053 |
| 28 | N-12 (257) | 603 | C-27 (390) | 1.596 |
| 29 | N-12 (257) | 603 | C-31 (394) | 1.540 |
| 30 | N-12 (257) | 603 | C-34 (397) | 1.844 |
| 31 | N-13 (258) | 603 | C-5 (368) | 0.849 |
| 32 | N-13 (258) | 603 | C-19 (382) | 1.418 |
| 33 | N-13 (258) | 603 | C-22 (385) | 1.440 |
| 34 | N-13 (258) | 603 | C-27 (390) | 1.768 |
| 35 | N-13 (258) | 603 | C-31 (394) | 1.889 |
| 36 | N-13 (258) | 603 | C-34 (397) | 1.328 |

BQ.1.1

| | | | | |
|---|---|---|---|---|
| R1 | N-12 (257) | 2045 | C-31 (394) | 2.730 |
| R2 | N-12 (257) | 2045 | C-34 (397) | 2.700 |
| R3 | N-13 (258) | 2045 | C-31 (394) | 2.535 |
| R4 | N-13 (258) | 2045 | C-34 (397) | 2.166 |

XBB

| | | | | |
|---|---|---|---|---|
| B1 | N-12 (257) | 2046 | C-31 (394) | 0.988 |
| B2 | N-12 (257) | 2046 | C-34 (397) | 1.271 |
| B3 | N-13 (258) | 2046 | C-31 (394) | 1.018 |
| B4 | N-13 (258) | 2046 | C-34 (397) | 1.214 |

TABLE 6

| SEQ | | |
|---|---|---|
| ID | | |
| NO: | Description | Sequence |
| 2045 | BQ.1.1 | VLYNFAPFFAFKCYGVSPTKLNDLCFTNVYADSFVIRGNEVSQIA |
| | Intervening | PGQTGNIADYNYKLPDDFTGCVIAWNSNKLDSTVGGNYNYRYRLF |
| | Sequence | RKSKLKPFERDISTEIYQAGNKPCNGVAGVNCYFPLQSYGFRPTY |
| | (AA) | GVGHQPYRVVVLSFELLHAPATVCGPKK |
| 2046 | XBB | VLYNFAPFFAFKCYGVSPTKLNDLCFTNVYADSFVIRGNEVSQIA |
| | Intervening | PGQTGNIADYNYKLPDDFTGCVIAWNSNKLDSKPSGNYNYRYRLF |
| | Sequence | RKSKLKPFERDISTEIYQAGNKPCNGVAGSNCYSPLQSYGFRPTY |
| | (AA) | GVGHQPYRVVVLSFELLHAPATVCGPKK |
| 2047 | BA.4/5 | VLYNFAPFFAFKCYGVSPTKLNDLCFTNVYADSFVIRGNEVSQIA |
| | Intervening | PGQTGNIADYNYKLPDDFTGCVIAWNSNKLDSKVGGNYNYRYRLF |
| | Sequence | RKSNLKPFERDISTEIYQAGNKPCNGVAGVNCYFPLQSYGFRPTY |
| | (AA) | GVGHQPYRVVVLSFELLHAPATVCGPKK |
| 2048 | BA.2.75 | VLYNFAPFFAFKCYGVSPTKLNDLCFTNVYADSFVIRGNEVSQIA |
| | Intervening | PGQTGNIADYNYKLPDDFTGCVIAWNSNKLDSKVSGNYNYLYRLF |
| | Sequence | RKSKLKPFERDISTEIYQAGNKPCNGVAGFNCYFPLQSYGFRPTY |
| | (AA) | GVGHQPYRVVVLSFELLHAPATVCGPKK |
| 2049 | BA 2.75.2 | VLYNFAPFFAFKCYGVSPTKLNDLCFTNVYADSFVIRGNEVSQIA |
| | Intervening | PGQTGNIADYNYKLPDDFTGCVIAWNSNKLDSKVSGNYNYLYRLF |
| | Sequence | RKSKLKPFERDISTEIYQAGNKPCNGVAGSNCYFPLQSYGFRPTY |
| | (AA) | GVGHQPYRVVVLSFELLHAPATVCGPKK |
| 2050 | BA 4.6 | VLYNFAPFFAFKCYGVSPTKLNDLCFTNVYADSFVIRGNEVSQIA |
| | Intervening | PGQTGNIADYNYKLPDDFTGCVIAWNSNKLDSKVGGNYNYRYRLF |
| | Sequence | RKSNLKPFERDISTEIYQAGNKPCNGVAGVNCYFPLQSYGFRPTY |
| | (AA) | GVGHQPYRVVVLSFELLHAPATVCGPKK |
| 2051 | BQ.1.1 | GTACTTTATAACTTCGCTCCCTTTTTCGCTTTCAAATGTTACGGA |
| | Intervening | GTGAGCCCTACAAAATTGAACGATCTCTGCTTCACCAACGTCTAT |
| | Sequence | GCAGACAGTTTTGTTATCCGTGGCAATGAAGTGAGCCAGATTGCA |
| | (Nucl.) | CCCGGTCAAACTGGGAACATTGCCGACTATAACTACAAACTTCCC |
| | | GACGACTTCACAGGGTGTGTAATCGCTTGGAACAGCAATAAACTC |
| | | GACTCTACCGTTGGAGGAAACTACAATTATCGCTACCGCCTTTTC |
| | | CGGAAGTCTAAACTCAAGCCCTTTGAGCGGGACATTAGCACTGAG |
| | | ATATATCAGGCAGGTAACAAACCATGCAATGGAGTAGCTGGTGTA |
| | | AACTGCTATTTCCCACTTCAGTCATACGGATTTAGACCCACATAC |
| | | GGTGTTGGACACCAACCATATCGTGTGGTTGTGCTCAGTTTTGAA |
| | | CTCCTTCATGCCCCTGCCACCGTGTGTGGGCCTAAAAAG |
| 2052 | XBB | GTTTTGTATAACTTCGCCCCCTTCTTCGCATTCAAGTGTTATGGC |
| | Intervening | GTCAGCCCAACCAAGCTTAATGACCTCTGTTTTACAAACGTCTAC |
| | Sequence | GCTGATAGCTTCGTGATACGTGGTAATGAAGTAAGTCAAATAGCC |
| | (Nucl.) | CCTGGCCAGACTGGTAACATTGCAGACTACAATTATAAACTTCCA |
| | | GATGACTTTACTGGGTGTGTAATCGCTTGGAATAGTAATAAGCTC |
| | | GACTCTAAGCCCTCCGGGAATTACAACTACCGATATCGGCTTTTC |
| | | AGAAAGTCTAAGCTCAAACCATTTGAGCGCGACATAAGCACAGAG |
| | | ATATATCAAGCAGGCAATAAGCCTTGTAACGGCGTAGCTGGTAGT |
| | | AATTGTTACTCCCCCCTGCAAAGTTATGGGTTCAGGCCAACTTAT |
| | | GGGGTGGGGCACCAGCCCTACAGAGTTGTCGTACTGAGCTTTGAG |
| | | CTGCTCCATGCTCCAGCTACAGTGTGTGGACCCAAGAAA |
| 2053 | BA.4/5 | GTGCTCTACAACTTCGCACCTTTCTTCGCTTTCAAGTGTTACGGT |
| | Intervening | GTTTCCCCCACTAAACTTAATGATCTGTGCTTTACTAACGTGTAC |
| | Sequence | GCAGACTCATTCGTTATTCGGGGTAACGAGGTTTCCCAAATCGCT |
| | (Nucl.) | CCCGGTCAAACCGGAAACATCGCTGATTACAACTATAAGTTGCCT |
| | | GATGACTTTACTGGGTGTGTCATCGCATGGAATTCCAACAAACTG |
| | | GATAGTAAAGTTGGAGGTAACTACAATTACCGATACAGGCTTTTC |
| | | CGTAAATCCAATCTGAAGCCCTTCGAGCGCGATATTAGTACTGAA |
| | | ATATATCAGGCTGGTAATAAACCTTGCAATGGGGTGGCTGGAGTA |
| | | AACTGCTACTTTCCTCTTCAGAGTTACGGGTTCAGGCCCACCTAT |
| | | GGAGTCGGACATCAACCATACCGTGTGGTTGTACTGTCATTTGAA |
| | | CTCCTTCACGCTCCCGCTACCGTGTGCGGCCCAAAGAAA |
| 2054 | BA.2.75 | GTTCTTTACAATTTCGCTCCCTTTTTCGCCTTCAAGTGTTATGGG |
| | Intervening | GTCTCTCCTACTAAACTCAATGACCTGTGCTTTACCAATGTGTAC |
| | Sequence | GCCGATTCCTTCGTTATACGAGGGAATGAGGTAAGTCAAATAGCC |
| | (Nucl.) | CCTGGACAAACCGGCAACATTGCTGATTACAATTACAAACTGCCT |
| | | GACGATTTCACCGGTTGTGTAATAGCCTGGAATTCCAATAAACTG |
| | | GATTCTAAAGTATCAGGCAACTACAACTATCTTTACAGGTTGTTT |
| | | AGAAAAAGCAAGCTGAAGCCCTTCGAGCGCGATATTTCAACAGAG |
| | | ATATATCAAGCAGGAAATAAACCATGCAACGGCGTCGCCGGCTTC |
| | | AATTGCTACTTCCCTTTGCAAAGTTATGGTTTTCGACCTACATAC |
| | | GGTGTCGGTCATCAGCCCTACCGCGTGGTCGTCCTGTCCTTTGAA |
| | | CTTCTTCACGCCCCAGCCACCGTATGTGGTCCCAAGAAG |
| 2055 | BA 2.75.2 | GTGCTCTACAATTTTGCACCCTTTTTCGCCTTCAAGTGTTACGGT |
| | Intervening | GTGAGCCCTACTAAGTTGAATGACCTCTGTTTTACAAATGTGTAC |
| | Sequence | GCTGACTCATTCGTGATACGTGGTAACGAAGTGAGTCAAATAGCC |
| | (Nucl.) | CCTGGGCAAACAGGTAACATCGCTGACTATAACTACAAGTTGCCC |
| | | GACGATTTTACAGGGTGTGTTATAGCATGGAACAGTAACAAGCTC |
| | | GATTCAAAGGTGAGCGGAAATTACAACTACCTCTATCGACTCTTT |
| | | AGGAAATCCAAGCTCAAGCCATTCGAGAGAGATATATCAACCGAA |
| | | ATTTACCAGGCTGGTAACAAGCCATGCAATGGGGTCGCAGGATCC |
| | | AACTGTTATTTCCCATTGCAGTCATACGGCTTCAGGCCCACTTAT |
| | | GGGGTAGGTCATCAACCCTACCGGGTCGTCGTCCTTTCTTTTGAA |
| | | CTTCTGCACGCACCAGCCACCGTATGCGGCCCCAAGAAG |
| 2056 | BA 4.6 | GTGCTCTATAACTTCGCTCCCTTCTTTGCTTTTAAATGCTACGGA |
| | Intervening | GTCTCCCCTACAAAGCTGAACGACCTTTGCTTCACTAATGTCTAC |
| | Sequence | GCCGATAGCTTTGTTATCAGAGGAAATGAAGTATCACAGATCGCA |
| | (Nucl.) | CCAGGTCAGACAGGCAACATCGCAGACTATAACTATAAACTTCCC |
| | | GACGATTTTACTGGCTGTGTCATTGCTTGGAACTCAAATAAACTC |
| | | GACTCAAAGGTGGGAGGAAATTACAACTATCGTTATAGGCTGTTC |
| | | AGGAAAAGTAATCTCAAGCCATTCGAGCGAGACATTAGTACAGAG |
| | | ATTTACCAGGCTGGAAACAAACCTTGCAATGGTGTCGCAGGCGTC |
| | | AACTGCTACTTCCCATTGCAGAGCTACGGCTTCAGGCCAACATAC |
| | | GGTGTGGGACACCAGCCTTACCGAGTTGTGGTCCTGTCCTTTGAA |
| | | CTTCTCCACGCACCTGCTACCGTCTGTGGACCCAAAAAA |
| 2057 | BA.X-1- | NITNLCPFDEVFNATKFASVYAWNRKRISNCVADYSVLYNSASFS |
| | monomer | AFKCYGVSPTKLNDLCFTNVYADSFVIRGNEVSQIAPGQTGNIAD |
| | (AA) | YNYKLPDDFTGCVIAWNSNKLDSKVGGNYNYRYRLFRKSNLKPFE |
| | | RDISTEIYQAGNKPCNGVAGENCYFPLRSYGFRPTYGVGHQPYRV |
| | | VVLSFELLHAPATVCGPKKSTNLVKNK |
| 2058 | BA.X-2- | NITNLCPFDEVFNATRFASVYAWNRKRISNCVADYSVLYNSAPFS |
| | monomer | AFKCYGVSPTKLNDLCFTNVYADSFVIRGNEVSQIAPGQTGNIAD |
| | (AA) | YNYKLPDDFTGCVIAWNSNKLDSKVGGNYNYLYRLFRKSNLKPFE |
| | | RDISTEIYQAGNKPCNGVAGENCYFPLRSYGFRPTYGVGHQPYRV |
| | | VVLSFELLHAPATVCGPKKSTNLVKNK |
| 2059 | BA.X-3- | NITNLCPFDEVFNATRFASVYAWNRKRISNCVADYSVLYNFAPFF |
| | monomer | AFKCYGVSPTKLNDLCFTNVYADSFVIRGNEVSQIAPGQTGNIAD |
| | (AA) | YNYKLPDDFTGCVIAWNSNKLDSKVGGNYNYRYRLFRKSNLKPFE |
| | | RDISTEIYQAGNKPCNGVAGENCYFPLRSYGFRPTYGVGHQPYRV |
| | | VVLSFELLHAPATVCGPKKSTNLVKNK |
| 2060 | BA.X-4- | NITNLCPFDEVFNATRFASVYAWNRKRISNCVADYSVLYNSASFS |
| | monomer | AFKCYGVSPTKLNDLCFTNVYADSFVIRGNEVSQIAPGQTGNIAD |
| | (AA) | YNYKLPDDFTGCVIAWNSNKLDSKVGGNYNYRYRLFRKSNLKPFE |
| | | RDISTEIYQAGNKPCNGVKGFNCYFPLRSYGFRPTYGVGHQPYRV |
| | | VVLSFELLHAPATVCGPKKSTNLVKNK |
| 2061 | BA.X-5- | NITNLCPFDEVFNATRFASVYAWNRKRISNCVADYSVLYNFAPFF |
| | monomer | AFKCYGVSPTKLNDLCFTNVYADSFVIRGNEVSQIAPGQTGNIAD |
| | (AA) | YNYKLPDDFTGCVIAWNSNKLDSKVGGNYNYRYRLFRKSNLKPFE |
| | | RDISTEIYQAGNKPCNGVKGFNCYFPLRSYGFQPTYGVGHQPYRV |
| | | VVLSFELLHAPATVCGPKKSTNLVKNK |
| 2062 | BA.X-6- | NITNLCPFDEVFNATRFASVYAWNRKRISNCVADYSVLYNSASFS |
| | monomer | AFKCYGVSPTKLNDLCFTNVYADSFVIRGNEVSQIAPGQTGNIAD |
| | (AA) | YNYKLPDDFTGCVIAWNSNKLDSKVGGNYNYLYRLFRKSNLKPFE |
| | | RDISTEIYQAGNKPCNGVAGENCYFPLQSYGFQPTYGVGYQPYRV |
| | | VVLSFELLHAPATVCGPKKSTNL VKNK |
| 2063 | BA.X-7- | NITNLCPFDEVFNATRFASVYAWNRKRISNCVADYSVLYNFAPFF |
| | monomer | AFKCYGVSPTKLNDLCFTNVYADSFVIRGNEVSQIAPGQTGNIAD |
| | (AA) | YNYKLPDDFTGCVIAWNSNKLDSKVGGNYNYLYRLFRKSNLKPFE |
| | | RDISTEIYQAGNKPCNGVAGENCYFPLRSYGFRPTYGVGHQPYRV |
| | | VVLSFELLHAPATVCGPKKSTNLVKNK |
| 2064 | BA.X-8- | NITNLCPFDEVFNATRFASVYAWNRKRISNCVADYSVLYNLASFS |
| | monomer | TFKCYGVSPTKLNDLCFTNVYADSFVIRGDEVRQIAPGQTGNIAD |
| | (AA) | YNYKLPDDFTGCVIAWNSNKLDSKVSGNYNYLYRLFRKSNLKPFE |
| | | RDISTEIYQAGNKPCNGVAGFNCYFPLRSYSFRPTYGVGHQPYRV |
| | | VVLSFELLHAPATVCGPKKSTNL VKNK |
| 2065 | BA.X-9- | NITNLCPFDEVFNATKFASVYAWNRKRISNCVADYSVLYNSASFS |
| | monomer | AFKCYGVSPTKLNDLCFTNVYADSFVIRGNEVSQIAPGQTGNIAD |
| | (AA) | YNYKLPDDFTGCVIAWNSNKLDSKVSGNYNYRYRLFRKSNLKPFE |
| | | RDISTEIYQAGNKPCNGVKGFNCYFPLRSYSFRPTYGVGHQPYRV |
| | | VVLSFELLHAPATVCGPKKSTNL VKNK |
| 2066 | BA.X-10- | NITNLCPFDEVFNATRFASVYAWNRKRISNCVADYSVLYNSAPFS |
| | monomer | AFKCYGVSPTKLNDLCFTNVYADSFVIRGNEVSQIAPGQTGNIAD |
| | (AA) | YNYKLPDDFTGCVIAWNSNKLDSKVGGNYNYRYRLFRKSNLKPFE |
| | | RDISTEIYQAGNKPCNGVAGFNCYFPLRSYGFRPTYGVGHQPYRV |
| | | VVLSFELLHAPATVCGPKKSTNLVKNK |
| 2067 | BA.X-1- | AATATCACAAATCTCTGCCCATTTGACGAAGTATTTAACGCCACC |
| | monomer | AAGTTTGCTTCAGTCTATGCTTGGAACCGGAAAAGGATTTCTAAT |
| | (Nucl.) | TGTGTAGCCGACTATAGTGTCCTCTACAATTCCGCCTCATTTTCA |
| | | GCCTTTAAATGCTACGGTGTATCACCTACTAAGCTCAACGATTTG |
| | | TGTTTTACAAACGTGTATGCTGATTCCTTTGTGATTCGTGGCAAC |
| | | GAAGTTTCTCAGATAGCCCCTGGTCAGACTGGCAACATTGCAGAT |
| | | TACAACTATAAACTGCCAGATGACTTTACTGGGTGTGTTATAGCC |
| | | TGGAATAGTAATAAACTTGACTCAAAAGTCGGTGGGAACTATAAC |
| | | TATCGTTATCGACTCTTTCGTAAATCAAACCTGAAGCCTTTCGAG |
| | | AGAGATATATCTACAGAGATTTACCAAGCTGGTAACAAACCTTGC |
| | | AACGGAGTAGCTGGCTTTAATTGTTATTTCCCTCTGCGTTCCTAT |
| | | GGTTTTCGACCAACCTACGGGGTGGGTCATCAGCCATACCGGGTC |
| | | GTAGTGCTGAGCTTCGAACTGTTGCACGCCCCTGCAACCGTGTGC |
| | | GGCCCTAAAAAGTCAACTAACCTGGTTAAGAATAAG |
| 2068 | BA.X-2- | AACATCACTAATCTGTGCCCTTTTGACGAGGTGTTCAACGCAACC |
| | monomer | CGATTTGCCTCAGTCTATGCATGGAACAGAAAGCGCATCAGTAAC |
| | (Nucl.) | TGCGTTGCCGATTATAGCGTTTTGTATAACTCTGCACCATTCTCT |
| | | GCATTCAAGTGTTATGGTGTGTCACCTACAAAGCTCAATGACCTT |
| | | TGTTTCACCAATGTGTATGCTGACTCTTTTGTGATCCGAGGGAAC |
| | | GAGGTAAGTCAGATTGCACCCGGACAGACCGGTAATATTGCTGAC |
| | | TACAATTACAAATTGCCAGATGACTTTACAGGATGCGTCATTGCT |
| | | TGGAACTCCAACAAGCTCGATAGCAAAGTGGGGGGGAATTATAAC |
| | | TATCTTTATCGACTGTTCCGTAAGTCAAATTTGAAACCATTTGAG |
| | | CGCGATATAAGTACAGAGATTTACCAGGCAGGGAACAAGCCTTGC |
| | | AACGGGGTCGCTGGTTTTAATTGTTACTTCCCTCTGAGGAGTTAT |
| | | GGATTTCGTCCAACATATGGTGTGGGCCATCAACCATACCGAGTT |
| | | GTAGTCCTTAGTTTCGAGTTGCTCCATGCTCCAGCCACAGTTTGT |
| | | GGCCCCAAAAAAAGCACCAACCTTGTAAAGAACAAG |
| 2069 | BA.X-3- | AACATTACCAACCTGTGTCCCTTCGACGAGGTCTTCAACGCAACA |
| | monomer | CGGTTCGCCTCAGTCTATGCTTGGAACCGAAAACGTATCTCCAAC |
| | (Nucl.) | TGCGTCGCAGACTATAGCGTCTTGTACAATTTCGCTCCTTTTTTC |
| | | GCATTTAAGTGCTACGGCGTGTCCCCAACTAAATTGAATGACTTG |
| | | TGTTTTACTAATGTGTATGCTGACTCCTTCGTAATTCGCGGTAAC |
| | | GAGGTATCCCAGATAGCACCTGGTCAGACCGGCAACATAGCCGAT |
| | | TATAACTACAAACTGCCTGATGATTTTACCGGATGTGTGATCGCT |
| | | TGGAATTCCAATAAACTTGACAGTAAAGTTGGAGGCAATTACAAT |
| | | TATCGCTATAGACTGTTCAGAAAAAGTAATTTGAAGCCTTTTGAA |
| | | AGAGACATTAGTACAGAGATATACCAAGCCGGAAATAAACCTTGT |
| | | AACGGCGTTGCAGGGTTTAATTGTTACTTCCCCCTGCGATCATAC |
| | | GGGTTTCGTCCAACCTATGGGGTAGGGCACCAACCTTACAGAGTA |
| | | GTTGTTCTGAGTTTTGAGCTCTTGCACGCACCTGCTACAGTTTGC |
| | | GGACCCAAGAAATCCACAAATTTGGTGAAGAATAAG |
| 2070 | BA.X-4- | AATATAACCAATCTCTGTCCTTTCGACGAAGTTTTCAACGCCACT |
| | monomer | CGGTTTGCCTCTGTGTATGCATGGAATCGAAAAAGAATTTCAAAT |
| | (Nucl.) | TGCGTGGCCGATTACTCTGTTCTTTACAACAGTGCATCCTTCTCC |
| | | GCTTTCAAATGTTATGGAGTGAGTCCAACCAAGCTTAATGATCTT |
| | | TGCTTCACCAACGTCTATGCAGACTCATTTGTTATACGTGGGAAT |
| | | GAAGTTTCTCAGATCGCCCCTGGACAGACCGGCAACATTGCAGAC |
| | | TACAATTACAAGCTGCCTGATGATTTTACAGGTTGCGTAATCGCC |
| | | TGGAACTCAAACAAGCTCGATTCCAAAGTAGGGGGGAATTACAAC |
| | | TACAGATACCGATTGTTTCGTAAAAGCAATCTGAAACCTTTTGAG |
| | | CGTGATATTTCTACTGAAATTTACCAAGCTGGAAATAAGCCTTGT |
| | | AATGGGGTTAAGGGATTTAATTGTTACTTCCCTTTGAGGTCTTAC |
| | | GGTTTCCGCCCCACTTATGGCGTAGGACACCAACCATATCGGGTG |
| | | GTGGTCCTTTCATTCGAGCTGCTGCACGCACCAGCAACTGTTTGC |
| | | GGACCAAAGAAGTCAACCAACCTGGTTAAAAATAAA |
| 2071 | BA.X-5- | AACATTACAAACCTGTGTCCTTTTGACGAGGTGTTCAATGCTACT |
| | monomer | CGGTTCGCATCCGTTTATGCATGGAACCGTAAACGCATCTCAAAT |
| | (Nucl.) | TGTGTCGCCGACTACTCAGTGCTCTACAACTTTGCCCCATTCTTT |
| | | GCATTTAAGTGTTACGGTGTATCACCCACCAAGCTCAACGACCTC |
| | | TGCTTCACTAACGTTTATGCCGATTCCTTTGTGATAAGGGGCAAC |
| | | GAAGTGAGCCAAATCGCCCCCGGACAAACTGGTAACATCGCCGAT |
| | | TATAATTATAAACTTCCCGACGACTTTACTGGGTGCGTGATAGCA |
| | | TGGAACTCAAACAAACTCGATTCCAAGGTCGGCGGTAATTATAAT |
| | | TATCGGTATAGGCTCTTCCGTAAGTCTAATCTCAAACCTTTCGAA |
| | | CGGGATATTTCAACAGAGATTTATCAGGCTGGGAATAAACCATGT |
| | | AACGGAGTAAAGGGCTTCAACTGTTACTTTCCCCTTCGAAGCTAC |
| | | GGTTTCCAGCCAACCTACGGGGTGGGCCACCAGCCCTACCGTGTA |
| | | GTCGTCCTTAGCTTCGAGTTGCTCCATGCCCCAGCCACTGTGTGC |
| | | GGACCAAAGAAGAGCACCAATCTTGTTAAGAATAAG |
| 2072 | BA.X-6- | AATATCACAAATCTCTGTCCTTTTGATGAGGTTTTTAATGCAACA |
| | monomer | CGCTTTGCCAGCGTCTATGCATGGAACCGGAAAAGAATAAGTAAT |
| | (Nucl.) | TGCGTAGCAGATTACTCTGTCCTCTATAATTCTGCATCATTCTCT |
| | | GCATTCAAATGTTATGGGGTTAGTCCCACAAAGTTGAACGATCTT |
| | | TGCTTCACCAACGTATATGCAGACAGCTTCGTCATTAGGGGGAAC |
| | | GAAGTATCACAGATCGCTCCCGGACAAACTGGGAACATAGCCGAC |
| | | TACAATTATAAGTTGCCTGATGACTTTACAGGTTGCGTCATTGCT |
| | | TGGAACTCAAATAAACTCGATTCCAAGGTCGGCGGCAATTATAAC |
| | | TATCTTTATAGGCTTTTCCGTAAGTCTAATCTGAAGCCTTTTGAG |
| | | AGAGATATATCTACTGAAATCTACCAGGCAGGTAATAAGCCTTGT |
| | | AACGGCGTAGCTGGTTTCAACTGTTATTTCCCCCTTCAATCATAT |
| | | GGTTTCCAACCAACCTACGGAGTCGGGTATCAGCCTTACCGCGTA |
| | | GTAGTTCTTAGTTTCGAGTTGCTTCATGCTCCAGCCACCGTGTGT |
| | | GGTCCTAAGAAATCAACAAATCTCGTCAAAAATAAA |
| 2073 | BA.X-7- | AACATCACAAACCTTTGTCCTTTTGATGAGGTATTTAATGCCACC |
| | monomer | AGGTTCGCATCTGTCTACGCATGGAATAGAAAACGGATCTCTAAT |
| | (Nucl.) | TGCGTTGCCGATTATAGCGTGCTTTACAACTTCGCTCCCTTCTTT |
| | | GCTTTTAAGTGCTATGGAGTCAGCCCAACAAAGCTCAATGATCTC |
| | | TGTTTCACAAATGTTTACGCAGATAGCTTTGTCATCCGCGGCAAC |
| | | GAGGTTTCCCAAATAGCACCAGGTCAGACTGGTAACATCGCAGAC |
| | | TATAACTATAAACTCCCAGACGATTTTACTGGGTGTGTTATTGCT |
| | | TGGAATAGCAATAAGTTGGACTCTAAGGTCGGTGGAAACTATAAT |
| | | TATTTGTATAGATTGTTCAGAAAAAGTAACCTCAAACCATTCGAG |
| | | AGAGACATTTCAACTGAGATATACCAAGCAGGTAACAAACCTTGC |
| | | AATGGGGTAGCAGGTTTTAATTGCTATTTCCCACTGCGAAGCTAT |
| | | GGTTTCCGTCCAACCTATGGTGTCGGCCATCAGCCTTACCGCGTA |
| | | GTGGTCCTCTCCTTTGAACTTCTTCACGCTCCTGCAACAGTTTGT |
| | | GGCCCCAAAAAGTCAACAAACCTCGTCAAAAATAAG |
| 2074 | BA.X-8- | AATATTACAAATCTCTGCCCATTTGACGAAGTCTTCAATGCAACC |
| | monomer | CGCTTCGCTTCAGTCTACGCCTGGAACCGCAAGCGCATCAGCAAC |
| | (Nucl.) | TGTGTAGCCGATTATAGTGTTCTCTATAATTTGGCCTCTTTCTCC |
| | | ACCTTCAAATGTTATGGTGTCAGCCCTACTAAACTTAACGACCTC |
| | | TGCTTTACCAACGTATATGCCGATTCATTCGTGATCCGTGGCGAT |
| | | GAGGTGCGCCAGATTGCCCCAGGGCAAACCGGCAACATAGCCGAC |
| | | TATAACTATAAGCTCCCCGATGACTTCACTGGTTGCGTAATCGCT |
| | | TGGAATTCCAATAAACTCGACTCCAAAGTCAGTGGTAACTATAAT |
| | | TACCTCTATCGCCTCTTTCGGAAATCAAATCTCAAGCCTTTCGAA |
| | | CGCGATATTTCCACAGAAATATACCAGGCAGGTAATAAGCCATGT |
| | | AACGGGGTCGCCGGGTTCAATTGCTATTTTCCTCTTAGGTCATAC |
| | | TCCTTTCGGCCCACTTATGGCGTGGGACACCAGCCTTACCGCGTT |
| | | GTAGTGCTTTCCTTTGAATTGCTGCATGCCCCAGCTACTGTCTGT |
| | | GGGCCTAAGAAGAGTACCAATCTTGTGAAGAATAAG |
| 2075 | BA.X-9- | AACATTACCAATCTGTGTCCTTTTGACGAAGTCTTCAATGCTACA |
| | monomer | AAATTTGCTAGCGTCTACGCATGGAACCGTAAACGAATAAGTAAC |
| | (Nucl.) | TGCGTGGCCGACTACTCTGTCTTGTATAACTCTGCCAGCTTCTCA |
| | | GCCTTCAAGTGCTATGGTGTTAGCCCAACCAAACTGAATGACCTG |
| | | TGTTTTACCAACGTCTACGCCGATTCCTTCGTCATCCGTGGTAAC |
| | | GAGGTCTCTCAAATAGCACCCGGACAAACAGGGAATATCGCAGAT |
| | | TACAATTATAAACTTCCTGATGACTTTACTGGCTGCGTGATCGCT |
| | | TGGAATAGCAATAAACTGGATTCTAAGGTGTCCGGCAACTATAAT |
| | | TACCGATATCGCCTCTTTCGCAAGTCTAATCTCAAACCCTTTGAA |
| | | CGCGACATCAGCACAGAAATCTATCAAGCTGGCAATAAGCCCTGC |
| | | AACGGGGTTAAGGGATTCAACTGTTATTTTCCCCTCAGGTCATAT |
| | | TCCTTTAGACCTACTTACGGTGTAGGCCATCAGCCCTATCGTGTG |
| | | GTAGTGCTTAGTTTCGAACTGCTTCACGCTCCCGCTACAGTTTGC |
| | | GGACCAAAAAAGTCTACCAATCTGGTTAAGAATAAA |
| 2076 | BA.X-10- | AACATCACAAACTTGTGCCCTTTTGATGAAGTGTTCAACGCTACT |
| | monomer | CGCTTTGCCTCAGTCTACGCTTGGAACAGAAAGCGGATAAGCAAT |
| | (Nucl.) | TGTGTCGCAGACTACTCCGTTCTCTATAACTCTGCCCCCTTTAGC |
| | | GCATTCAAATGTTACGGAGTGTCTCCTACTAAACTTAACGATCTC |
| | | TGCTTTACAAACGTATATGCTGATTCCTTCGTGATTAGGGGGAAC |
| | | GAGGTCTCCCAAATTGCACCCGGCCAGACCGGGAACATCGCAGAC |
| | | TATAACTACAAGCTGCCTGATGATTTTACTGGATGCGTTATCGCC |
| | | TGGAATAGCAACAAACTCGACAGTAAAGTTGGCGGGAACTACAAC |
| | | TACAGATACCGTCTTTTTCGGAAAAGCAATCTCAAACCTTTTGAA |
| | | AGAGACATATCAACAGAAATATATCAGGCAGGCAATAAACCTTGT |
| | | AATGGAGTAGCTGGATTCAACTGTTACTTTCCACTTCGGAGTTAC |
| | | GGGTTTCGTCCAACATATGGTGTCGGGCACCAACCTTATCGTGTA |
| | | GTAGTGCTCAGTTTTGAACTTCTTCACGCTCCAGCTACCGTGTGT |
| | | GGTCCTAAGAAATCCACCAATCTGGTGAAGAACAAG |

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A recombinant polypeptide comprising at least one immunogenic fragment of Severe Acute Respiratory Syndrome Coronavirus 2 (SARS-CoV-2) spike glycoprotein, wherein each immunogenic fragment comprises an N-terminus region, wherein the N-terminus region comprises an amino acid sequence selected from the group consisting of SEQ ID NOs: 245-289 and 293-362, and amino acid sequences DYS, YS, and S.

2. The recombinant polypeptide of claim 1, wherein each immunogenic fragment further comprises a C-terminus region, and the C-terminus region comprises an amino acid sequence selected from the group consisting of SEQ ID NOs: 363-398 and 402-423, and amino acid sequences STN, ST, and S.

3. A recombinant polypeptide comprising at least one immunogenic fragment of SARS-CoV-2 spike glycoprotein, wherein each immunogenic fragment comprises an N-terminus region and a C-terminus region, and wherein the N-terminus region comprises an amino acid sequence that differs at at least 2 residues from an amino acid sequence selected from the group consisting of SEQ ID NOs: 245-289 and 293-362, DYS, YS, and S, and the C-terminus region comprises an amino acid sequence that differs at at least 2 residues from an amino acid sequence selected from the group consisting of SEQ ID NOs: 363-398 and 402-423, STN, ST, and S.

4. A recombinant polypeptide comprising at least one immunogenic fragment of SARS-CoV-2 spike glycoprotein, wherein each immunogenic fragment comprises an N-terminus region and a C-terminus region, and wherein the N-terminus region comprises an amino acid sequence of at least five contiguous amino acid residues of an amino acid sequence selected from the group consisting of SEQ ID NOs: 245, 293, 314, and 342, and the C-terminus region comprises an amino acid sequence of at least five contiguous amino acid residues of an amino acid sequence of SEQ ID NO: 363 or 402.

5. The recombinant polypeptide of claim 4, wherein each immunogenic fragment comprises an N-terminus region and a C-terminus region, and wherein the N-terminus region comprises an amino acid sequence of at least ten contiguous amino acid residues of an amino acid sequence selected from the group consisting of SEQ ID NOs: 245, 293, 314, and 342 and the C-terminus region comprises an amino acid sequence of at least ten contiguous amino acid residues of an amino acid sequence of SEQ ID NO: 363 or 402.

6. The recombinant polypeptide of claim 2, wherein each immunogenic fragment comprises an intervening region between the N-terminus region and the C-terminus region, and wherein each intervening region comprises an amino acid sequence selected from the group consisting of SEQ ID NOs: 2045-2050 or that differs at at least 4 residues from an amino acid sequence selected from the group consisting of SEQ ID NOs: 603-606, and 2037-2040.

7. The recombinant polypeptide of claim 1, wherein each of the at least one immunogenic fragments comprises an amino acid sequence with at least 95% sequence identity to an amino acid sequence independently selected from the group consisting of SEQ ID NOs: 1577-1661 and 1917-1946.

8. The recombinant polypeptide of claim 1, wherein the polypeptide comprises at least two immunogenic fragments.

9. The recombinant polypeptide of claim 1, wherein each immunogenic fragment of the at least one immunogenic fragments comprises the same amino acid sequence.

10. The recombinant polypeptide of claim 8, wherein each immunogenic fragment of the at least one immunogenic fragment comprises a different amino acid sequence from each of the other immunogenic fragments.

11. The recombinant polypeptide of claim 1, wherein the recombinant polypeptide is expressed at a concentration of at least 45 mg protein/L culture.

12. The recombinant polypeptide of claim 1, wherein the recombinant polypeptide exhibits at least 85% protease resistance.

13. The recombinant polypeptide of claim 1, wherein the recombinant polypeptide does not comprise an amino acid sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 217, 219, 221, 223, 225, 227, 229, 231, 233, 235, 237, 239, 241, 243, and 2057-2066.

14. The recombinant polypeptide of claim 1, wherein the recombinant polypeptide further comprises an antibody Fc region.

15. The recombinant polypeptide of claim 1, wherein the polypeptide comprises at least two immunogenic fragments, and wherein the at least two immunogenic fragments are connected to each other via a linker.

16. The recombinant polypeptide of claim 15, wherein the linker is a polypeptide comprising an amino acid sequence of 1-35 residues, wherein each residue is independently serine or glycine.

17. The recombinant polypeptide of claim 14, wherein the at least one immunogenic fragment of the SARS-CoV-2 spike glycoprotein is connected to the antibody Fc region via a linker.

18. The recombinant polypeptide of claim 17, wherein the linker comprises an amino acid sequence selected from the group consisting of SEQ ID NOs: 65 (Fc1), SEQ ID NO: 67 (Fc1-TEV), SEQ ID NO: 69 (Fc1-Rv3C), SEQ ID NO: 193 (Short), SEQ ID NO: 195 (Medium), and SEQ ID NO: 197 (Long).

19. The recombinant polypeptide of claim 14, wherein the antibody Fc region is from a human IgG1 antibody or derived therefrom.

20. The recombinant polypeptide of claim 19, wherein the antibody Fc region comprises the amino acid sequence of SEQ ID NO: 71.

21. The recombinant polypeptide of claim 1, wherein the polypeptide comprises an amino acid sequence with at least 95% sequence identity to an amino acid sequence selected from the group consisting of SEQ ID NOs: 1662-1746 and 1947-1976.

22. The recombinant polypeptide of claim 21, wherein the polypeptide comprises an amino acid sequence selected from the group consisting of SEQ ID NOs: 1662-1746 and 1947-1976.

23. A recombinant polynucleotide encoding the polypeptide of claim 1.

24. The recombinant polynucleotide of claim 23, wherein the polynucleotide comprises a nucleic acid sequence with at least 93% sequence identity to a nucleic acid sequence selected from the group consisting of SEQ ID NOs: 1832-1916 and 2007-2036.

25. The recombinant polynucleotide of claim 24, wherein the polynucleotide comprises a nucleic acid sequence selected from the group consisting of SEQ ID NOs: 1832-1916 and 2007-2036.

26. The recombinant polynucleotide of claim 23, wherein the nucleic acid sequence has been codon optimized.

27. A pharmaceutical composition comprising the recombinant polypeptide of claim 1 and a pharmaceutically acceptable carrier.

28. The pharmaceutical composition of claim 27, wherein the pharmaceutical composition further comprises at least one adjuvant, and
wherein the at least one adjuvant is selected from the group consisting of alum adjuvants, emulsion adjuvants, and pattern recognition receptor agonist adjuvants, or
wherein the at least one adjuvant is AS03, MF59, CpG, Squalene Emulsion, Alum, aluminum hydroxide gels, calcium phosphate hydroxide, paraffin oil, cytokines (IL-1, IL-2, IL-12), killed bacterial products such as Bordetella and Mycobacterium bacteria, bacterial toxoids, squalene and DL-α-tocopherol emulsions, aluminum phosphate gels, saponins, cyclic dinucleotides, and TLR agonists, and any combination thereof, or
wherein the at least one adjuvant is a squalene-oil-in-water emulsion adjuvant.

29. The pharmaceutical composition of claim 27, wherein the pharmaceutical composition does not comprise an adjuvant.

30-32. (canceled)

33. A vector comprising the recombinant polynucleotide of claim 23.

34. An isolated cell comprising the polypeptide encoded by the recombinant polynucleotide of claim 23.

35. A method for preventing, inhibiting, reducing, eliminating, protecting, or delaying the onset of an infection or an infectious clinical condition caused by a beta coronavirus in a subject comprising administering to the subject the recombinant polypeptide of claim 1.

36. A method for inducing an immune response against a beta coronavirus in a subject comprising administering to the subject the recombinant polypeptide of claim 1.

37. The method of claim 35, wherein the recombinant polypeptide is administered by oral, parenteral, subcutaneous, intradermal, transdermal, intravenous, intramuscular, intranasal, intrapulmonary, intraarterial, intrathecal, or interperitoneal administration.

38. The method of claim 35, wherein the coronavirus is selected from the group consisting of SARS-CoV-2, SARS-CoV, and MERS-CoV

39. The method of claim 35, wherein the subject is a mammal.

40. The method of claim 39, wherein the mammal is a human or non-human primate.

41-42. (canceled)
